(19) United States
(12) Patent Application Publication
(10) Pub. No.: US 2023/0348953 A1
(43) Pub. Date: Nov. 2, 2023

(54) HIGH THROUGHPUT, FLUORESCENCE-BASED ESTERASE ACTIVITY ASSAY FOR ASSESSING POLYSORBATE DEGRADATION RISK DURING BIOPHARMACEUTICAL DEVELOPMENT

(71) Applicant: GENENTECH, INC., South San Francisco, CA (US)

(72) Inventors: Li YI, South San Francisco, CA (US); Adithi Chandrasekhara BHARGAVA, South San Francisco, CA (US); Inn Huam YUK, South San Francisco, CA (US)

(73) Assignee: GENENTECH, INC., South San Francisco, CA (US)

(21) Appl. No.: 18/043,568

(22) PCT Filed: Aug. 31, 2021
(86) PCT No.: PCT/US2021/048526

§ 371 Date: Feb. 28, 2023

Related U.S. Application Data
(60) Provisional application No. 63/218,103, filed on Jul. 2, 2021.
(60) Provisional application No. 63/140,592, filed on Jan. 22, 2021.
(60) Provisional application No. 63/119,451, filed on Nov. 30, 2020.
(60) Provisional application No. 63/072,656, filed on Aug. 31, 2020.

(51) Int. Cl. C12Q 1/44

(52) U.S. Cl. CPC C12Q 1/44; G01N 2333/918

(57) ABSTRACT

The present disclosure provides compositions, methods, and kits for detecting lipolytic activity. In some embodiments, the composition comprises an aqueous assay sample and an organic solvent, wherein the organic solvent comprises 4-methylumbellif-erryl caprylate (MU-C8). Also provided herein are methods for determining the stability of a protein preparation.

### BACKGROUND

Protein-based biotherapeutics have had outstanding success in treating severe diseases, such as various forms of cancer and immune-mediated disorders. However, despite the technical advancements in biotherapeutic manufacturing over the last few decades, parenteral protein formulations are restricted to a small number of commonly deployed surfactants, each bearing their own characteristic shortcomings and challenges. Polysorbates (PS) represent the most common class of surfactants in biopharmaceutical formulations, and have set the benchmark in terms of protein stabilizing properties, biocompatibility and safety. The most commonly used polysorbates, PS20 and PS80, consist of a core of dehydrated sorbitol, i.e. a mixture of sorbitan and isosorbide that is polyethoxylated and esterified with fatty acids, mainly lauric acid in PS20 and oleic acid in PS80. Degradation of polysorbates can occur through oxidation or chemical hydrolysis, leading to the accumulation of free fatty acids (FFAs). Degradation over time (during the shelf-life of the drug product) is a concern because it may result in (i) the occurrence of visible particulates due to the insoluble matter of polysorbate degradants; (ii) an adverse impact on protein quality; (iii) in reduced concentration of surfactant, leading to insufficient protection of the protein against interfacial stress; and (iv) potential differences in the drug product’s safety profile.

A major contributor to PS degradation are host cell proteins (HCPs). Protein based therapeutics are often produced by expressing the therapeutic protein in mammalian or microbial cell cultures. Protein formulations are prepared by isolating the expressed target protein from the cell culture supernatant. In addition to expressing the therapeutic protein, these cell cultures produce their own naturally occurring proteins-i. e. HCPs, which can contaminate the protein formulation and hydrolyze polysorbates. Downstream purification processes remove a large majority of the HCPs found in the supernatant with the therapeutic protein; however, trace amounts of HCP typically remain. Examples of HCP proteins sometimes identified in protein formulations and associated with hydrolytic degradation include lysosomal phospholipase A2 (LPLA2), putative phospholipase B-like 2 (PLBL2), lipoprotein lipase (LPL), liver carboxylesterase B-1 like (CES-B1L), and liver carboxylesterase 1-like (CES-1L). The full extent and identity of degradation causing HCPs in a given protein formulation is difficult to measure, because the amounts of hydrolytic enzymes (HCPs) in a typical drug product (DP) are miniscule and PS degradation may be attributed to one or more enzymes with high catalytic activity.

A variety of methods have been developed to detect polysorbate degradation. High performance liquid chromatography (HPLC) coupled with evaporative light scattering detector (ELSD) provides a useful tool for measuring and quantifying intact polysorbate in solution. A shallow gradient, reversed-phase chromatography method equipped with ELSD allows for the separation and qualitative assessment of polysorbate esters and sub-species, but not robust quantitation. Recently, the development and implementation of a FFA assay has been reported, which can quantify fatty acids found in polysorbate solutions using reversed-phase ultra high performance liquid chromatography (UHPLC) equipped with photodiode array (PDA) detector. Additional mass spectrometry methods have also been reported to quantify products of polysorbate degradation. These methods are time-intensive and do not have the sensitivity to detect changes in polysorbate content until a sufficient level of degradation has occurred.

Recently developed methods for detection of esterase activity use fluorogenic or chromogenic substrates that have been modified to contain a fatty acid side chain to mimic the polysorbate ester bond. Acyloxymethyl ethers and 1-acyloxy-1-cyano-3-proplyl ethers of umbelliferone have been identified as stable fluorescent substrates for esterase and lipase detection due to their secondary reaction with periodate and bovine serum albumin. Endpoint and kinetic assays using 4-methylumbelliferone (MU) have also been reported. Finally, commercially available kits, like EnzChekTM, have also been reported to be highly sensitive against LPL and can be useful tool for monitoring lipase activity.

Trace amounts of hydrolytic enzymes present in protein therapeutics solutions can be difficult to detect. Improved methods for the rapid high-throughput detection of these enzymes are therefore needed.

### SUMMARY

In various embodiments, the present disclosure relates to an assay for determining enzymatic activity of host cell proteins (HCPs) in a sample, wherein the HCPs comprises hydrolase, the assay comprising the steps of: obtaining a reaction mixture in a microplate, wherein the reaction mixture comprises: the sample, a reaction buffer, and a 4-methylumbelliferone carboxylate ester as a fluorogenic substrate; obtaining a negative control; exposing the reaction mixture and the negative control to fluorescence signal; monitoring conversion of the fluorogenic substrate from a non-fluorescent state to a fluorescent product in the reaction mixture resulting from exposure to the fluorescence signal, wherein the fluorescent product is 4-methylumbelliferone (MU); and determining and quantifying the HCP enzymatic activity based on conversion of the fluorogenic substrate.

In various embodiments, the sample comprises two or more different HCPs. In various embodiments, the HCP enzymatic activity represents the collective activity of two or more HCPs in the sample. In various embodiments, the reaction mixture comprises at least two different fluorogenic substrates. In various embodiments, the HCPs include esterases. In various embodiments, the HCPs include carboxylic ester hydrolases, and wherein the HCPs optionally include lipases and carboxylesterases. In various embodiments, the HCPs the fluorogenic substrate has a carbon chain length of 8, 10, 12, 16 and/or 18. In various embodiments, the fluorogenic substrate is 4-methylumbelliferyl caprylate (MU-C8). In various embodiments, the fluorogenic substrate is 4-methylumbelliferyl decanoate (MU-C10).

In various embodiments, the sample comprises a product from a prokaryotic or eukaryotic host. In various embodiments, the sample comprises a recombinant protein produced by a prokaryotic or eukaryotic host. In various embodiments, the sample comprises a recombinant protein produced by bacterial or mammalian host. In various embodiments, the sample comprises a recombinant protein produced by a prokaryotic or eukaryotic host. In various embodiments, the sample comprises a recombinant protein that is based on an IgG format and is produced by a bacterial or mammalian host. In various embodiments, the sample comprises a recombinant protein that is based on an IgG format and is produced by an E.coli or a Chinese Hamster Ovary (CHO) host.

In various embodiments, the sample comprises a recombinant protein selected from the group consisting of an IgG1 mAb, an IgG4 mAb, a bi-specific antibody; a mAb produced by a bacterial host, and a mAb produced by a mammalian host.

In various embodiments, the negative control is an enzyme blank. In various embodiments, the fluorogenic substrate in the reaction mixture has a concentration of about 0.1-5 mM, about 0.1-4 mM, about 0.1-3 m M, about 0.1-2 mM, or about 0.5-1.0 mM. In various embodiments, the sample is a chromatography purified pool sample. In various embodiments, the sample is exposed to an increase of fluorescence signal using excitation and emission wavelengths of 300-400 nm and 400-500 nm, respectively, optionally about 355 nm and 460 nm, respectively. In various embodiments, the sample is being incubated, optionally for about 1-5 hours, about 1-4 hours, about 1-3 hours, or about 2 hours. In various embodiments, the sample is monitored every 5-15 minutes, or wherein the sample is optionally monitored every 10 minutes. In various embodiments, the reaction mixture has a pH of about 4-9, about 5-9, about 6-9, about 7-9, or about 8.

In various embodiments, the enzymatic activity is used to assess the level of hydrolytic activity towards polysorbate degradation in the sample. In various embodiments, an output of the assay is used to compare and select purification processes to improve removal of hydrolytic HCPs.

In various embodiments, the present disclosure relates to an assay for determining enzymatic activity of host cell proteins (HCPs) in a sample, wherein the HCPs comprises hydrolase, and the assay comprises the steps: (a) obtaining a reaction mixture comprising the sample, a reaction buffer, and a fluorogenic substrate, wherein the fluorogenic substrate is 4-methylumbelliferone carboxylate ester, wherein the carboxylate ester of the 4-methylumbelliferone carboxylate ester comprises no more than ten carbons; (b) measuring the fluorescent signal at one or more time points; and (c) determining and quantifying the HCP enzymatic activity based on the measured fluorescence. In various embodiments, the carboxylate ester of the 4-methylumbelliferone carboxylate ester comprises no more than 8 carbons.

In various embodiments, the 4-methylumbelliferone carboxylate ester is MU-C8. In various embodiments, the 4-methylumbelliferone carboxylate ester is MU-C10. In various embodiments, the HCP enzymatic activity determined and quantified in step c) represents the collective activity of two or more HCPs in the sample. In various embodiments, the assay further comprises obtaining a negative control comprising the same reaction buffer and fluorogenic substrate as the reaction mixture; measuring the fluorescent signal of the negative control at the same one or more time points; and determining and quantifying the HCP enzymatic activity by subtracting the amount of fluorescent signal observed in the negative control from the amount of fluorescent signal observed in the reaction mixture. In various embodiments, the reaction mixture comprises at least two different fluorogenic substrates. In various embodiments, the HCPs include esterases. In various embodiments, the HCPs include carboxylic ester hydrolases, optionally the HCPs include lipases and carboxylesterases.

In various embodiments, the sample comprises a product from a prokaryotic or eukaryotic host. In various embodiments, the sample comprises a recombinant protein produced by a prokaryotic or eukaryotic host. In various embodiments, the sample comprises a recombinant protein produced by bacterial or mammalian host. In various embodiments, the sample comprises a recombinant protein that is based on an IgG format and is produced by a bacterial or mammalian host. In various embodiments, the sample comprises a recombinant protein that is based on an IgG format and is produced by an E.coli or a Chinese Hamster Ovary (CHO) host. In various embodiments, the sample comprises a recombinant protein being selected from the group consisting of an IgG1 mAb, an IgG4 mAb, a bi-specific antibody; a mAb produced by a bacterial host, and a mAb produced by a mammalian host.

In various embodiments, the negative control is an enzyme blank. In various embodiments, the fluorogenic substrate in the reaction mixture has a concentration of about 0.1-5 mM, about 0.1-4 mM, about 0.1-3 m M, about 0.1-2 mM, or about 0.5-1.0 mM. In various embodiments, the sample is a chromatography purified pool sample. In various embodiments, in step b), the sample is exposed to an increase of fluorescence signal using excitation and emission wavelengths of 300-400 nm and 400-500 nm, respectively, optionally about 355 nm and about 460 nm, respectively. In various embodiments, in step c), the sample is incubated, optionally for about 2 hours, about 1-5 hours, about 1-4 hours, or about 1-3 hours. In various embodiments, in step c), the sample is monitored every 5-15 minutes, or wherein the sample is optionally monitored every 10 minutes. In various embodiments, the reaction mixture has a pH of about 4-9, about 5-9, about 6-9, about 7-9, or about 8. In various embodiments, the enzymatic activity is used to assess the level of hydrolytic activity towards polysorbate degradation in the sample. In various embodiments, an output of the assay is used to compare and select purification processes to improve removal of hydrolytic HCPs.

In various embodiments, the present disclosure relates to a composition comprising (a) an aqueous assay sample comprising a protein preparation, (b) an organic solvent comprising a reaction buffer, and at least one 4-methylumbelliferone carboxylate ester; wherein the fluorogenic substrate is 4-methylumbelliferone carboxylate ester, and wherein the carboxylate ester of the 4-methylumbelliferone carboxylate ester comprises no more than ten carbon atoms.

In various embodiments, the present disclosure relates to a method of determining stability of a protein preparation comprising (a) obtaining a reaction mixture in a microplate, wherein the reaction mixture comprises: the sample, a reaction buffer, and a 4-methylumbelliferone carboxylate ester as a fluorogenic substrate; (b) obtaining a negative control; (c) exposing the reaction mixture and the negative control to a fluorescence signal; (d) monitoring conversion of the fluorogenic substrate from a non-fluorescent state to a fluorescent product in the reaction mixture resulting from exposure to the fluorescence signal, wherein the fluorescent product is 4-methylumbelliferone (MU); and (e) determining and quantifying the HCP enzymatic activity based on conversion of the fluorogenic substrate in step (d).

In various embodiments, the present disclosure relates to a method of optimizing or selecting a protein purification process to improve removal of hydrolytic HCPs, said method comprising (a) obtaining a reaction mixture in a microplate, wherein the reaction mixture comprises: the sample, a reaction buffer, and a 4-methylumbelliferone carboxylate ester as a fluorogenic substrate; (b) obtaining a negative control; (c) exposing the reaction mixture and the negative control to a fluorescence signal; (d) monitoring conversion of the fluorogenic substrate from a non-fluorescent state to a fluorescent product in the reaction mixture resulting from exposure to the fluorescence signal, wherein the fluorescent product is 4-methylumbelliferone (MU); and (e) determining and quantifying the HCP enzymatic activity based on conversion of the fluorogenic substrate in step (d).

### BRIEF DESCRIPTION OF THE DRAWINGS

FIGS. 1A-1E show chemical structures of substrates used in assay development: a) 4-methylumbelliferyl caprylate (MU-C8); b) 4-methylumbelliferyl decanoate (MU-C10); c) 4-methylumbelliferyl dodecanoate (MU-C12); d) 4-methylumbelliferyl palmitate (MU-C16); e) 4-methylumbelliferyl oleate (MU-C18:1).

FIG. 2 shows esterase activity rates and substrate specificities of three model enzymes and three purified protein samples to 4-methylumbelliferone substrates of different carbon chain lengths. PCL (20 ng/ml), LPLA2 (20 ng/ml), PLBL2 (400 µg/ml) and purified protein samples (neat, mAbs 1-3, 150-225 mg/ml) were tested. Results are reported as an average of duplicate samples from a single plate; error bars represent ±1 SD from mean.

FIGS. 3A-3B show typical fluorescence time course profiles for the esterase activity assay using MU-C8 as the fluorogenic substrate. FIG. 3A depicts fluorescence signal over time from MU-C8 incubation in the presence and absence of purified mAb 2. The fluorescence in the presence of purified mAb 2 represents the total amount of hydrolysis (both enzymatic and non-enzymatic) and is used to calculate k_{raw} and the fluorescence in the absence of mAb 2 (enzyme blank) represents nonenzymatic hydrolysis (background fluorescence) and is used to calculate k_{non-enzymatic} _{hydrolysis}. FIG. 3B depicts a standard curve of MU fluorescence over incubation time at varying MU concentrations (right). Results are reported as an average of duplicate samples from a single plate; error bars represent ±1 SD from mean.

FIG. 4 shows orlistat incubation and corresponding esterase activity rates measured using MU-C8 substrate for model enzymes PCL and LPLA2 (50 ng/ml in orlistat incubation) and purified mAb 1 (25 mg/ml in orlistat incubation). Results are reported as an average of duplicate samples or controls from a single plate. Error bars represent ±1 SD from mean.

FIGS. 5A-5C show impact of pH on esterase activity assay. pH dependence of MU fluorescence in reaction buffer without substrate (top left): results are reported as an average of three measurements across three independent plate preparations. pH dependence of non-enzymatic hydrolysis of MU-C8 substrate in reaction buffer (top right): results are reported as an average of five measurements across three independent plate preparations. pH dependence of esterase activity rates for model enzyme PCL and purified mAb 2 (bottom): results for PCL are reported as an average of three measurements across three independent plate preparations; results for mAb 2 are reported as an average of duplicate measurements across eight plates in total (one plate per pH level was tested). The pH values shown represent measurements taken from MU, MU-C8 or sample wells immediately after completing the experiments. All error bars represent ±1 SD from mean.

FIGS. 6A-6B show changes in fluorescence (top) and non-enzymatic hydrolysis (bottom) with different sample matrices in the presence of fluorogenic substrate (MU-C8). Changes were calculated relative to control (water). Results are reported as an average of duplicate well measurements; error bars represent ±1 SD from mean.

FIGS. 7A-7C show studies on effect of proteins and excipients on esterase activity assay. Esterase activity rates based on MU-C8 hydrolysis were calculated at different concentrations of LPLA2 in the presence or absence of mAb 4 at 180 mg/ml (top left). MU fluorescence was measured in the presence (50, 100, and 200 mg/mL) or absence of mAb 1 at varying MU concentrations (0, 5 and 10 µM) (top right). Esterase activity rates of model enzyme PCL, purified mAb 1 (neat) and purified mAb 3 (neat) were measured for control samples (no spike), or samples spiked with 0.1% PS20 or 0.1% PS80 (bottom). Results are reported as an average of duplicate well measurements; error bars represent ±1 SD from mean.

FIGS. 8A-8B show a comparison of three purification processes used for mAb 5 for their effects on esterase activities rates and polysorbate degradation. The samples tested were taken from UFDF pools generated by three different purification schemes used for mAb 5 (Process A, B and C). The purified samples were incubated with fluorogenic MU-C8 substrate to assess the esterase activity rates (left): results are reported as an average of duplicate wells; error bars represent ±1 SD from mean. The purified samples were formulated with PS20 and the PS20 content for each sample was measured before and after incubation at 40° C. for 7 days by the HPLC-ELSD method (right): results are reported as percent decrease in PS20 content measured from an average of duplicate HPLC injections.

FIGS. 9A-9B show the correlation between rates of PS80 degradation and esterase activity for mAb 2 samples. Rates of PS80 degradation by C18:1 FFA release were determined using FFA assay (left). Rates of PS80 degradation by PS80 content decrease were determined using HPLC-ELSD method (right). PS80 degradation was measured in samples taken before and after incubation at 25° C. for 42 days. Esterase activity rates were measured for the same samples (without 42-day incubation) using MU-C8 substrate. Samples tested were sourced from two different purification process schemes applied to CHO (Chinese Hamster Ovary) cell culture harvests; these mAb 2 samples covered multiple stages in the purification process (affinity chromatography, ion-exchange chromatography, and UFDF). Results are reported as an average of duplicate wells; error bars represent ±1 SD from mean.

### DETAILED DESCRIPTION

The present disclosure relates to compositions, methods, and kits for detecting enzymatic activity, for detecting polysorbate degradation and for determining the stability of protein formulations. A fast, high-throughput, fluorescence-based esterase activity assay has now been developed and is useful as a quantification and a risk assessment tool for bioprocess development. Further provided herein are compositions comprising an aqueous assay sample comprising a protein preparation and an organic solvent, which comprises a reaction buffer and at least one fluorogenic substrate, wherein the fluorogenic substrate is a 4-methylumbelliferone carboxylate ester, and wherein the carboxylate ester of the 4-methylumbelliferone carboxylate ester comprises no more than ten carbons. The compositions and methods provided here are further useful for determining the stability of a protein preparation, and/or for optimizing or selecting a protein purification process to improve the removal of hydrolytic HCPs.

The esterase activity rates measured by this assay disclosed herein have been demonstrated to correlate with polysorbate degradation rates. In various embodiments, the increased esterase activity-measured by the hydrolysis of the carboxylic ester bond in the fluorogenic substrate-corresponds with decreased PS20 content, decreased PS80 content and/or increased FFA. The methods disclosed herein provide a suitable assay for assessing the risk of polysorbate degradation during bioprocess development in a rapid, high-throughput manner. The assays disclosed herein have been found to be sensitive, broadly applicable and compatible with a wide variety of purified protein and purification in-process pool sample types and matrices.

In various embodiments, a method of measuring enzymatic activity of a host cell protein in a protein sample is provided. Such assays may involve obtaining a reaction mixture comprising the protein sample, a reaction buffer and a fluorogenic substrate. Nonlimiting examples of representative protein samples, reaction buffers and fluorogenic substrates are provided in detail below. In various embodiments, the fluorescent signal emitted from the reaction mixture is measured over time at one or more time points. One of the benefits of the various embodiments disclosed herein is that the assay provides for rapid, high throughput determination of enzymatic activity. In various embodiments, the fluorescence is measured at 0 hours, .1 hours, .2 hours, .3 hours, .4 hours, .5 hours, .6 hours .7 hours, .8 hours, .9 hours, 1 hour, 1.1 hours, 1.2 hours, 1.3 hours, 1.4 hours, 1.5 hours, 1.6 hours, 1.7 hours, 1.8 hours, 1.9 hours, 2 hours, 2.5 hours, and/or three hours. In various embodiments, the fluorescence is measured continuously over a 30 minute, 1 hour, 1.5 hour, 2 hour, 2.5 hour or 3 hour time period.

In various embodiments, the amount of fluorescence emitted from the reaction mixture represents the amount of HCP enzymatic activity present in the reaction mixture. In various embodiments, however, the amount of fluorescence may also represent non-enzymatic activity, and a negative control may be necessary. In various embodiments, an enzyme blank reaction (i.e., negative control) is obtained for each reaction mixture/protein sample, with identical buffer matrix to the sample but omitting the protein from the solution. In various embodiments, this blank reaction measures non-enzymatic hydrolysis of the substrate and is subtracted from the total hydrolytic activity measured in the reaction mixture/protein sample to derive the enzymatic activity of the sample. Enzyme blank reaction rate represents the non-enzymatic hydrolysis rate of the substrate in a specific sample matrix (k_{non-enzymatic} _{hydrolysis}). Some sample backgrounds (e.g. acetate, histidine, arginine and sulfate) altered the fluorescence signal of the MU reaction product or affected the rate of non-enzymatic hydrolysis. Therefore, it is important to subtract the background fluorescence from the overall fluorescence signal in calculating the esterase activity rates. By offering a total turnaround time of under 3 hours that is considerably shorter than traditional surfactant spiking and incubation methods and other activity assays reported to date, this assay can support more rapid evaluation of polysorbate degradation risks during biopharmaceutical development.

In some embodiments, a control sample is measured in parallel with an aqueous assay sample for lipolytic activity. In some embodiments, the present disclosure provides a method of detecting enzymatic activity in an aqueous assay sample, the method comprising (a) combining the aqueous assay sample comprising a protein preparation, with an organic solvent comprising a 4-methylumbelliferyl carboxylate ester (4Mu) to form an assay composition; (b) combining a control sample comprising a protein preparation and a lipase inhibitor, with an organic solvent comprising 4-methylumbelliferyl carboxylate ester (4Mu) to form a control composition; and (c) measuring the formation of carboxylate ester and 4-methylumbelliferone (4Mu) by fluorescence in the assay composition and in the control composition. In some embodiments, the protein preparation of (a) and the protein preparation of (b) are provided from the same protein preparation. For example, a protein preparation from a cell culture is obtained from which two aliquots can be removed. One aliquot can be the protein preparation of the aqueous assay sample, and the other aliquot can be the protein preparation of the control sample. In some embodiments, the protein preparation of (a) and the protein preparation of (b) comprise substantially the same components. In some embodiments, the protein preparation of (a) and the protein preparation of (b) are expected to have the same level of enzymatic activity. In some embodiments, the aqueous assay sample and the control sample have substantially the same components except for the lipase inhibitor in the control sample. In some embodiments, the control sample comprising the lipase inhibitor is a negative control sample, i.e., no fluorescence is expected to be detected. In some embodiments, the method further utilizes a positive control sample, i.e., wherein fluorescence is expected. In some embodiments, a positive control sample comprises a known quantity of 4Mu. In some embodiments, a positive control sample comprises a known quantity of 4-methylumbelliferyl carboxylate ester and a known quantity of an active enzyme.

As discussed herein, an enzyme having hydrolytic activity can interfere with components of a protein preparation. In some embodiments, an enzyme having hydrolytic activity hydrolyzes fatty acids and/or esters present in a protein preparation. In some embodiments, an enzyme having hydrolytic activity hydrolyzes a surfactant present in a protein preparation. In some embodiments, hydrolysis of the surfactant reduces stability of the protein preparation. By measuring the amount of hydrolytic activity using the methods provided herein, the level of hydrolysis that has occurred in the protein preparation can then be determined based on the measured amount of hydrolytic activity, thereby determining the stability of the protein preparation. In some embodiments, the present disclosure provides a method of determining stability of a protein preparation, comprising (a) combining an aqueous assay sample comprising a protein preparation with an organic solvent comprising a 4-methylumbelliferone carboxylate ester; (b) measuring the formation of carboxylate ester and 4-methylumbelliferone (4Mu) by fluorescence; and (c) determining the stability of the protein preparation based on the measured fluorescence. For example, increased fluorescence, relative to a control, would indicate the presence of enzymatic activity, indicating that excipients, e.g., surfactants such as polysorbate, have been hydrolyzed, thereby forming non-polar, and therefore insoluble, long chain fatty acids that may destabilize the protein in the protein preparation. In some embodiments, the method is used to determine the stability of a protein preparation for a pharmaceutical formulation.

### Protein Preparations

In various embodiments, the invention disclosed herein relates to a method of detecting enzymatic activity of host cell proteins in or determining the stability of a protein preparation that has undergone purification. As used herein in the context of protein preparations, “purification” refers to a process in which one or more substances, e.g., proteins, are isolated from a complex mixture, typically cells, tissues, or organisms. A “purified” protein sample or protein preparation can refer to a sample in which one or more non-water-soluble components of a cell, tissue, or organism (such as, e.g., cell membranes, lipids, aggregated proteins or nucleic acids, and other hydrophobic substances) have been reduced or removed and leaving only the soluble components (such as, e.g., soluble proteins). As used herein, “soluble” can refer to the ability of a substance to dissolve in a certain solvent, e.g., a cell culture medium, a buffer, water or an organic solvent. In the context of proteins, “soluble” can also refer to proteins that do not precipitate and/or aggregate in a certain solvent, e.g., a cell culture medium, a buffer, water, or an organic solvent.

An exemplary purification process can include: growing a cell culture containing the protein of interest, e.g., a therapeutic protein; separating the cells from the culture media; lysing the cells and separating the lysed cells to generate a cell culture supernatant containing the soluble components and a pellet containing the insoluble components described herein; and subjecting the cell culture supernatant to buffer exchange, pH adjustment, centrifugation, filtration (including, e.g., ultrafiltration and/or diafiltration), chromatography, or any combination thereof to generate a purified protein preparation. In some embodiments, a purified protein preparation of the present disclosure is purified by the process described herein. In some embodiments, a partially purified protein preparation of the present disclosure has been subjected to part of the purification process described herein. For example, a partially purified protein preparation may not have been subjected to all of the buffer exchange, pH adjustment, centrifugation, filtration, and/or chromatography steps used for generating the purified protein preparation. In some embodiments, a cell culture supernatant described herein includes a therapeutic protein of the present disclosure. In some embodiments, a partially purified protein preparation described herein includes a therapeutic protein of the present disclosure. In some embodiments, a purified protein preparation described herein includes a therapeutic protein of the present disclosure.

As used herein, “aqueous” (e.g., aqueous assay sample) refers to a solution or sample in which water is the solvent. Thus, aqueous assay samples of the present disclosure can include, cell culture media, buffer solutions, protein samples, and the like. In some embodiments, the aqueous assay sample of the present disclosure comprises a protein preparation.

In some embodiments, the protein preparation is a cell culture supernatant. Cell culture supernatants are described herein and can be obtained, e.g., from a cell culture for producing a protein of interest. In some embodiments, the protein is a therapeutic protein. In some embodiments, a cell culture supernatant is produced after lysing the cultured cells and separating the soluble and insoluble components, e.g., by centrifugation. Examples of cells and cell lines suitable for culturing and protein production are provided herein. In some embodiments, the cell culture supernatant comprises a protein of interest, e.g., a therapeutic protein, and additional host cell components. In some embodiments, the additional host cell components comprise additional host cell proteins with enzymatic activity. In some embodiments, the additional host cell proteins comprises a lipase. In some embodiments, the lipase has lipolytic activity.

In some embodiments, the protein preparation is a partially purified protein preparation. Partially purified protein preparations are described herein and can be obtained, e.g., after undergoing a partial purification procedure (e.g., a purification process described herein) for a protein of interest from a cell culture. In some embodiments, the protein is a therapeutic protein. In some embodiments, a partially purified protein preparation has undergone additional purification steps compared to a cell culture supernatant. In some embodiments, a partially purified protein preparation comprises a therapeutic protein and additional components of the host cell. In some embodiments, the host cell components comprise host cell proteins. In some embodiments, the host cell proteins comprise a lipase. In some embodiments, the lipase has lipolytic activity. In some embodiments, the therapeutic protein is 20% to 95% (w/w), 30% to 90% (w/w) or 40% to 80% (w/w) of all proteins in the in the partially purified protein preparation.

In some embodiments, the protein preparation is a purified protein preparation. Purified protein preparations are described herein and can be obtained, e.g., after undergoing a purification procedure (e.g., a purification process described herein) for a protein of interest from a cell culture. In some embodiments, the protein of interest is a therapeutic protein. In some embodiments, a purified protein preparation a therapeutic protein and additional components of the host cell. In some embodiments, the host cell components comprise host cell proteins. In some embodiments, the host cell proteins comprises a lipase. In some embodiments, the lipase has lipolytic activity. In some embodiments, the therapeutic protein is greater than 70% (w/w), greater than 80% (w/w), greater than 85% (w/w), greater than 90% (w/w), greater than 95% (w/w), or greater than 99% (w/w) of all proteins in the in the purified protein preparation.

In some embodiments, the protein preparation comprises a therapeutic protein. Non-limiting examples of therapeutic proteins include antibodies (such as monoclonal or polyclonal antibodies) and antibody fragments; protein-based vaccines (such as, e.g., hepatitis B surface antigen); blood factors (such as, e.g., Factor VIII and Factor IX); thrombolytic agents (such as, e.g., tissue plasminogen activator); hormones (such as, e.g., insulin, glucagon, growth hormone, and gonadotrophin); hematopoietic growth factors (such as, e.g., erythropoietin and colony stimulating factors); interferons (such as, e.g., interferon-α, interferon-β, and interferon-v); interleukin-based proteins (such as, e.g., interleukin-12); and other proteins such as tumor necrosis factor and therapeutic enzymes. Further examples of protein therapeutics are described in, e.g., Dimitrov, Methods Mol Biol 899: 1-26 (2012), Lagasse et al., F1000Res 6: 113 (2017), and Protein Therapeutics, Eds: Vaughan et al., 2017: Wiley-VCH Verlag. Therapeutic proteins can include recombinant proteins, modified proteins and fusion proteins, such as, e.g., antibody-drug conjugates, antibody-cytokine fusions, Fc-fusions, bispecific antibodies, multispecific antibodies, affibody fusions, glycosylated proteins and peptides, and engineered receptor antagonists. In some embodiments, the protein preparation comprising the therapeutic protein is used in a pharmaceutical formulation.

In some embodiments, the protein preparation comprises a commercially important protein, e.g., an industrial enzyme. Commercially important proteins can be used in a variety of industries such as pharmaceuticals, chemical production, biofuels, food and beverage, and consumer products. For example, in some embodiments, the protein preparation is an enzyme used within a process to generate a desired product or may be the product of interest. In some embodiments, the commercially important protein is used in the food, pharmaceutical synthesis, biofuel, chemical, or manufacturing industries. In some embodiments, the industrial enzyme includes, but is not limited to, palatase lipozyme, lipopan, xylose isomerase, bromelain and noopazyme (used in the food industry), cellulase and amylase (used in the biofuel industry), resinase (used in the paper processing industry), amidase (used in the chemical industry), novozym-435 (used in cosmetic production of isopropyl myristate) or subtilisin (used in detergents).

In some embodiments, the protein preparation comprises a pharmaceutical excipient. Pharmaceutical excipients are included, e.g., to aid in the processing of the drug delivery system before, during, or after manufacture; to protect, support, or enhanced stability, bioavailability, or patient acceptability; to assist in product identification and enhance overall safety; to assist in the effectiveness and/or delivery of the drug in use; and/or to assist in maintaining integrity of the drug product during storage. Non-limiting examples of pharmaceutical excipients include surfactants, fillers, diluents, binders, suspending agents, viscosity agents, coatings, flavoring agents, disintegrants, colorants, lubricants, glidants, preservatives, sweeteners, and the like. In some embodiments, the pharmaceutical excipient is added to the protein preparation. In some embodiments, the pharmaceutical excipient is added to the protein preparation before, after, or during purification.

In some embodiments, a protein preparation comprising a therapeutic protein is purified, then stored for a period of time (e.g., for less than 4 hours, less than 8 hours, less than 1 day, about 1 day, about 2 days, about 3 days, about 4 days, about 5 days, about 6 days, about 1 week, greater than 1 week, about 2 weeks, greater than 2 weeks, about 3 weeks, greater than 3 weeks, about 1 month, greater than 1 month, about 2 months, greater than 2 months, about 3 months, or greater than 3 months). The protein preparation is then subjected to the present method to detect lipolytic activity.

In some embodiments, the pharmaceutical excipient is a surfactant. As used herein, “surfactant” refers to an agent that lowers surface tension or interfacial tension between two liquids. In some embodiments, surfactants can stabilize a composition, e.g., a protein preparation described herein, by minimizing aggregation and/or precipitation and/or improving solubility (e.g., by lowering surface tension and inhibiting protein surface adsorption; see, e.g., Agarkhed et al., AAPS PharmSciTech 14:1-9 (2013)) of one or more components of the composition. Surfactants in pharmaceutical compositions can also modulate bioavailability of an active pharmaceutical ingredient (API); assist the API in maintaining a preferred polymorphic form; prevent aggregation or dissociation; and/or modulate immunogenic responses of active ingredients. Surfactants can include cationic, anionic, non-ionic, zwitterionic, amphoteric, and/or ampholytic surfactants. Non-limiting examples of surfactants include polysorbates (e.g., TWEEN^{®} surfactants such as TWEEN^{®} 20 and TWEEN^{®} 80, which are also known as polysorbate 20, polysorbate 80, respectively) derived from ethoxylated sorbitans esterified with fatty acids (e.g., lauric acid in polysorbate 20 and oleic acid in polysorbate 80); tyloxapols; poloxamers (e.g., PLURONIC^{®} F68LF, PLURONIC^{®} L-G2LF, PLURONIC^{®} L62D, LUTROL^{®} F68, and KOLLIPHOR^{®} P188); polyoxyethylene castor oil (e.g., KOLLIPHOR^{®} EL) and derivatives thereof; sorbitan esters, also known as Spans; polyoxyl stearates; lecitins; phospholipids; polyoxyethylene surfactants such as, e.g., TRITON^{®} (e.g., TRITON^{®} X-100) and BRIJ^{®} (e.g., BRIJ^{®} 35); and polyethoxylated fatty acids such as, e.g., MYRJ^{®} S40, MYRJ^{®} S100, and MYRJ^{®} 52.

In some embodiments, the surfactant comprises a fatty acid. In some embodiments, the surfactant comprises an ester. In some embodiments, the surfactant is a polysorbate. Polysorbates are a class of compounds derived from ethoxylated sorbitans esterified with fatty acids and include, e.g., polysorbate 20, polysorbate 40, polysorbate 60, polysorbate 80, polysorbate 21, polysorbate 61, polysorbate 65, polysorbate 81, and polysorbate 81. In some embodiments, the protein preparation provided herein includes a polysorbate. In some embodiments, the polysorbate in the protein preparation is polysorbate 20, polysorbate 80, or combinations thereof.

In some embodiments, the surfactant is at about 0.001% w/v to about 2% w/v of the aqueous assay sample. In some embodiments, the surfactant is at about 0.005% w/v to about 2% w/v of the aqueous assay sample. In some embodiments, the surfactant is at about 0.01% w/v to about 2% w/v of the aqueous assay sample. In some embodiments, the surfactant is at about 0.02% to about 1.5% w/v of the aqueous assay sample. In some embodiments, the surfactant is at about 0.03% to about 1.0% w/v of the aqueous assay sample. In some embodiments, the surfactant is at about 0.04% to about 0.8% w/v of the aqueous assay sample. In some embodiments, the surfactant is at about 0.05% to about 0.6% w/v of the aqueous assay sample. In some embodiments, the surfactant is at about 0.06% to about 0.4% w/v of the aqueous assay sample. In some embodiments, the surfactant is at about 0.07% to about 0.2% w/v of the aqueous assay sample. In some embodiments, the surfactant is at about 0.08% to about 0.15% w/v of the aqueous assay sample. In some embodiments, the surfactant is at about 0.09% to about 0.10% w/v of the aqueous assay sample. In some embodiments, the surfactant is at about 0.01% to about 0.04% w/v of the aqueous assay sample. In some embodiments, the surfactant is a polysorbate. In some embodiments, the surfactant is polysorbate 20, polysorbate 80, or combinations thereof.

### Enzymatic Activity of Host Cell Proteins

In some embodiments, the protein preparation further comprises one or more additional host cell proteins. As described herein, the protein preparation is prepared from a cell culture, i.e., comprising host cells of the protein of interest, e.g., a therapeutic protein. In some embodiments, the protein preparation includes one or more additional host cell proteins. In some embodiments, the additional host cell proteins are soluble in substantially the same conditions as the protein of interest, e.g., therapeutic protein. In some embodiments, the additional host cell proteins are not easily separable from the protein of interest, e.g., therapeutic protein. In some embodiments, the additional host cell proteins comprise a lipase. In some embodiments, one or more of the additional host cell proteins have lipolytic activity.

In some embodiments, the present disclosure relates to compositions and methods for the detection of lipolytic activity. Lipolytic activity, i.e., lipolysis, generally refers to the hydrolysis of lipids. The lipolysis reaction can be catalyzed by lipase enzymes, which is a subclass of esterase enzymes. Thus, “lipase” refers to an enzyme that hydrolyzes ester bonds of a lipid, e.g., a triglyceride, a phospholipid, a cholesteryl ester, and the like. Lipases include, e.g., triglyceride lipase, lipoprotein lipase, pancreatic lipase, hepatic lipase, gastric lipase, lingual lipase, endothelial lipase, and phosphatidylserine phospholipase. Lipases are produced naturally, e.g., produced by the pancreas, liver, lingual glands, stomach, thyroids, and/or mucosa in mammals, secreted by certain bacteria and fungi, and/or found in the lysosome. In some embodiments, the lipase is endogenous to the cell from which the protein in the protein purification was derived. In some embodiments, the lipase is endogenous to another biological component in the protein preparation, e.g., a biological component comprising a stabilizing protein added to the protein preparation.

As referred to herein, “active” lipases are lipases capable of performing lipolysis (also referred to herein as having “lipolytic activity”). Active lipases present in a protein preparation can interfere with downstream processes involving the protein of interest, e.g., therapeutic protein. In some embodiments, a protein preparation comprising a protein of interest, e.g., therapeutic protein, and a lipase is included in a pharmaceutical formulation. In some embodiments, the excipient is added to the protein preparation. In some embodiments, the excipient stabilizes the protein preparation, e.g., by minimizing interfacial stress, reducing protein aggregation, and/or improving protein solubility. In some embodiments, the excipient is a surfactant. In some embodiments, the excipient comprises a fatty acid, an ester, or both. In some embodiments, the excipient is prone to hydrolysis by an active lipase. In some embodiments, the presence of an active lipase in a protein preparation comprising the protein of interest and excipient reduces stability of the preparation. It is therefore advantageous to reliably detect lipolytic activity in a protein preparation, in order to minimize the negative impacts, such as increased particles, safety concerns (due to, e.g., increased injection site reactions), and decreased quality, caused by lipase hydrolysis of excipients.

In some embodiments, lipases are produced by cells in a cell culture. In some embodiments, lipases are produced by cells in a cell culture for the production of a protein of interest. Non-limiting examples of cells suitable for production of a protein of interest include bacterial, insect, yeast, mammalian, and/or transgenic cells. Non-limiting examples of cell lines include CHO, HEK 293, HT-1080, PER.C6, CAP, VERO, BHK, HeLa, CV1, Cos, MDCK, 3T3, NS0, NS1, PC12, W138, Sp2/0, HKB-11, TM4, MMT 060562, TR1, MRC 5, FS4, myeloma cell lines, hybridoma cell lines, and hepatoma cell lines. In some embodiments, the cell line for producing the protein of interest is a stable cell line, e.g., wherein the gene for the protein of interest is stably integrated into the genome of the cell. In some embodiments, the cell line for producing the protein of interest is a transient cell line, e.g., wherein the cells express, but do not integrate the gene into the genome.

### Fluorogenic Substrates

In various embodiments, the present invention relates to an enzymatic assay that utilizes a model esterase substrate, 4-methylumbelliferyl carboxylate ester, which is composed of the fluorescent dye 4-methylumbelliferone (MU) esterified to a carboxylate acid. The fluorogenic substrate is quenched while intact, but the fluorescence can be detected when the carboxylic ester bond is cleaved to release MU. The carboxylate ester can be of any carbon length. In preferred embodiments the carboxylate ester comprises less than 12, less than 11, less than 10, less than 9, less than 8, less than 7, less than 6, less than 5, less than 4, less than 3, or less than 2 esters. In various embodiments, the fluorogenic substrate is 4-methylumbelliferyl caprylate (MU-C8), which is composed of the fluorescent dye 4-methylumbelliferone (MU) esterified to caprylic acid, an eight carbon, saturated fatty acid. In various embodiments, the fluorogenic substrate is MUC-C10.

In some embodiments, fluorescence of 4Mu is used to detect the hydrolysis of the 4-methylumbelliferyl carboxylate ester by a hydrolytic enzyme, and thus is an indicator of hydrolytic activity. The skilled artisan will understand that the hydrolysis of 4-methylumbelliferyl carboxylate ester (as measured by 4Mu fluorescence) will likely indicate that hydrolytic activity has occurred in the protein preparation, i.e., that surfactants may have been hydrolyzed, possibly rendering the protein preparation unstable. In some embodiments, fluorescence of 4Mu can be measured at excitation wavelength of about 330 nm and emission wavelength of about 495 nm. In some embodiments, fluorescence of 4Mu can be measured at excitation wavelength of about 327 nm and emission wavelength of about 449 nm. In some embodiments, fluorescence of 4Mu can be measured at excitation wavelength of about 300 nm to about 350 nm and emission wavelength of about 420 nm to about 500 nm. In some embodiments, fluorescence measurement parameters of 4Mu (e.g., the excitation and emission wavelengths) vary when pH is changed. In some embodiments, fluorescence measurement parameters of 4Mu vary when salt and/or buffering agent concentration is changed. In some embodiments, of 4-methylumbelliferyl carboxylate ester is a substrate for a lipase. In some embodiments, of 4-methylumbelliferyl carboxylate ester is hydrolyzed by a lipase in the protein preparation described herein. In some embodiments, 4Mu formation is measured by fluorescence. In some embodiments, hydrolytic activity of an assay sample comprising a protein preparation described herein, is measured by the fluorescence of 4Mu.

### Buffers and Reaction Conditions

In some embodiments, the aqueous assay sample comprising the protein preparation further comprises a buffering agent, a salt, or both. In general, a salt of the present disclosure refers to an ionic compound whose anion is not OH⁻ and O²⁻. In some embodiments, the salt reduces and/or prevents degradation of one or more components in the composition. Suitable salts that can be included in an aqueous assay samples can be selected by one of ordinary skill in the art, including, e.g., sodium salts, potassium salts, calcium salts, ammonium salts, and the like. In some embodiments, the salt is potassium chloride (KCl), sodium chloride (NaCl), sodium carbonate (Na₂CO₃), sodium sulfate (Na₂SO₄), calcium chloride (CaCl₂), ammonium chloride (NH₄Cl), ammonium acetate (NH₄CH₃COO), ammonium sulfate ((NH₄)₂SO₄), or combination thereof. In some embodiments, the salt is NaCl, CaCl₂, or combination thereof. In some embodiments, the salt is both NaCl and CaCl₂.

In some embodiments, the concentration of NaCl in the aqueous assay sample facilitates accurate and/or efficient detection of lipolytic activity in the sample. In some embodiments, the NaCl is about 10 mM to about 500 mM in the aqueous assay sample. In some embodiments, the NaCl is about 25 mM to about 400 mM in the aqueous assay sample. In some embodiments, the NaCl is about 50 mM to about 300 mM in the aqueous assay sample. In some embodiments, the NaCl is about 75 mM to about 250 mM in the aqueous assay sample. In some embodiments, the NaCl is about 100 mM to about 200 mM in the aqueous assay sample. In some embodiments, the NaCl is about 50 mM, about 60 mM, about 70 mM, about 80 mM, about 90 mM, about 100 mM, about 110 mM, about 120 mM, about 130 mM, 140 mM, 150 mM, 160 mM, 170 mM, 180 mM, 190 mM, or 200 mM in the aqueous assay buffer.

In some embodiments, the NaCl is about 10 mM to about 500 mM in the final composition (aqueous assay sample and organic solvent). In some embodiments, the NaCl is about 25 mM to about 400 mM in the final composition. In some embodiments, the NaCl is about 50 mM to about 300 mM in the final composition. In some embodiments, the NaCl is about 75 mM to about 250 mM in the final composition. In some embodiments, the NaCl is about 100 mM to about 200 mM in the final composition. In some embodiments, the NaCl is about 100 mM to about 140 mM, e.g., 120 mM in the final composition. In some embodiments, the NaCl is about 50 mM, about 60 mM, about 70 mM, about 80 mM, about 90 mM, about 100 mM, about 110 mM, about 120 mM, about 130 mM, 140 mM, 150 mM, 160 mM, 170 mM, 180 mM, 190 mM, or 200 mM in the final composition.

In some embodiments, the concentration of CaCl₂ in the aqueous assay sample facilitates accurate and/or efficient detection of lipolytic activity in the sample. In some embodiments, the CaCl₂ is about 0.1 mM to about 20 mM in the aqueous assay sample. In some embodiments, the CaCl₂ is about 0.2 mM to about 10 mM in the aqueous assay sample. In some embodiments, the CaCl₂ is about 0.5 mM to about 5.0 mM in the aqueous assay sample. In some embodiments, the CaCl₂is about 0.7 mM to about 3.0 mM in the aqueous assay sample. In some embodiments, the CaCl₂ is about 1.0 mM to about 2.0 mM in the aqueous assay sample. In some embodiments, the CaCl₂ is about 0.5 mM, about 0.6 mM, about 0.7 mM, about 0.8 mM, about 0.9 mM, about 1.0 mM, about 1.1 mM, about 1.2 mM, about 1.3 mM, about 1.5 mM, about 1.5 mM, about 1.6 mM, about 1.7 mM, about 1.8 mM, about 1.9 mM, about 2.0 mM, about 2.5 mM, about 3.0 mM, about 3.5 mM, about 4.0 mM, about 4.5 mM, or about 5.0 mM in the aqueous assay sample.

In some embodiments, the CaCl₂ is about 0.1 mM to about 20 mM in the final composition (aqueous assay sample and organic solvent). In some embodiments, the CaCl₂ is about 0.2 mM to about 10 mM in the final composition. In some embodiments, the CaCl₂ is about 0.5 mM to about 5.0 mM in the final composition. In some embodiments, the CaCl₂ is about 0.7 mM to about 3.0 mM in the final composition. In some embodiments, the CaCl₂ is about 1.0 mM to about 2.0 mM in the final composition. In some embodiments, the CaCl₂ is about 0.5 mM, about 0.6 mM, about 0.7 mM, about 0.8 mM, about 0.9 mM, about 1.0 mM, about 1.1 mM, about 1.2 mM, about 1.3 mM, about 1.5 mM, about 1.5 mM, about 1.6 mM, about 1.7 mM, about 1.8 mM, about 1.9 mM, about 2.0 mM, about 2.5 mM, about 3.0 mM, about 3.5 mM, about 4.0 mM, about 4.5 mM, or about 5.0 mM in the final composition.

In some embodiments, the NaCl and CaCl₂ reduce and/or prevent degradation of one or more components in the aqueous assay sample. In some embodiments, the NaCl and CaCl₂ reduce and/or prevent aggregation and/or precipitation of the protein, e.g., the therapeutic protein. In some embodiments, the NaCl and CaCl₂ reduce and/or prevent degradation of one or more components in the composition that is not in the aqueous assay sample, e.g., in the organic solvent. In some embodiments, the NaCl and CaCl₂ reduce and/or prevent autohydrolysis of 4-methylumbelliferyl carboxylate ester. In some embodiments, the NaCl is about 10 mM to about 500 mM, and the CaCl₂ is about 0.1 mM to about 20 mM in the aqueous assay sample. In some embodiments, the NaCl is about 25 mM to about 400 mM, and the CaCl₂ is about 0.2 mM to about 10 mM in the aqueous assay sample. In some embodiments, the NaCl is about 50 mM to about 300 mM, and the CaCl₂ is about 0.5 mM to about 5.0 mM in the aqueous assay sample. In some embodiments, the NaCl is about 75 mM to about 250 mM, and the CaCl₂ is about 0.7 to about 3.0 mM in the aqueous assay sample. In some embodiments, the NaCl is about 100 mM to about 200 mM, and the CaCl₂ is about 1.0 to about 2.0 mM in the aqueous assay sample. In some embodiments, the NaCl is about 150 mM and the CaCl₂ is about 0.3 mM in the aqueous assay sample.

As used herein, a “buffering agent” refers to a substance used in a solution to maintain the pH of the solution. Buffering agents can maintain a solution at a certain pH range (i.e., the buffering capacity in a given range) and prevent a rapid change in pH when additional components are added to the solution. In general, a buffering agent can be a weak acid or weak base. In some embodiments, the buffering agent has a buffering capacity at about pH 5.0, about pH 5.5, about pH 6.0, about pH 6.5, or about pH 7.0. Buffering agents with buffering capacity of about pH 5.0 to about pH 7.0 include, e.g., citrate, acetate, phosphate, MES, Bis-Tris, ADA, ACES, PIPES, MOPSO, Bis-Tris propane, BES, MOPS, TES, HEPES, DIPSO, MOBS, TAPSO, and Tris. The buffering capacity of a buffering agent can be determined by the skilled artisan. In some embodiments, the buffering agent in the aqueous assay sample facilitates accurate and/or efficient detection of lipolytic activity in the sample. In some embodiments, the buffering agent reduces and/or prevents degradation of one or more components in the aqueous assay sample. In some embodiments, the buffering agent reduces and/or prevents aggregation of the protein, e.g., the therapeutic protein. In some embodiments, the buffering agent reduces and/or prevents degradation of one or more components in the composition that is not in the aqueous assay sample, e.g., in the organic solvent of the composition. In some embodiments, the buffering agent reduces and/or prevents autohydrolysis of the 4-methylumbelliferyl carboxylate ester. In some embodiments, the buffering agent is provided in the aqueous assay sample as an aqueous buffer solution.

In some embodiments, the buffering agent is about 5 mM to about 200 mM in the aqueous assay sample. In some embodiments, the buffering agent is about 10 mM to about 100 mM in the aqueous assay sample. In some embodiments, the buffering agent is about 20 mM to about 80 mM in the aqueous assay sample. In some embodiments, the buffering agent is about 30 mM to about 70 mM in the aqueous assay sample. In some embodiments, the buffering agent is about 40 mM to about 60 mM in the aqueous assay sample. In some embodiments, the buffering agent is about 10 mM, about 12 mM, about 15 mM, about 18 mM, about 20 mM, about 22 mM, about 25 mM, about 28 mM, about 30 mM, about 32 mM, about 35 mM, about 38 mM, about 40 mM, about 42 mM, about 45 mM, about 48 mM, about 50 mM, about 52 mM, about 55 mM, about 58 mM, about 60 mM, about 62 mM, about 65 mM, about 68 mM, about 70 mM, about 72 mM, about 75 mM, about 78 mM, about 80 mM, about 82 mM, about 85 mM, about 88 mM, about 90 mM, about 92 mM, about 95 mM, about 98 mM, or about 100 mM in the aqueous assay sample. In some embodiments, the buffering agent is Bis-Tris. In some embodiments, the buffering agent is Tris.

In some embodiments, the buffering agent is about 5 mM to about 200 mM in the final composition (aqueous assay sample and organic solvent). In some embodiments, the buffering agent is about 10 mM to about 100 mM in the final composition (aqueous assay sample and organic solvent). In some embodiments, the buffering agent is about 20 mM to about 80 mM in the final composition. In some embodiments, the buffering agent is about 30 mM to about 70 mM in the final composition. In some embodiments, the buffering agent is about 40 mM to about 60 mM in the final composition. In some embodiments, the buffering agent is about 10 mM, about 12 mM, about 15 mM, about 18 mM, about 20 mM, about 22 mM, about 25 mM, about 28 mM, about 30 mM, about 32 mM, about 35 mM, about 38 mM, about 40 mM, about 42 mM, about 45 mM, about 48 mM, about 50 mM, about 52 mM, about 55 mM, about 58 mM, about 60 mM, about 62 mM, about 65 mM, about 68 mM, about 70 mM, about 72 mM, about 75 mM, about 78 mM, about 80 mM, about 82 mM, about 85 mM, about 88 mM, about 90 mM, about 92 mM, about 95 mM, about 98 mM, or about 100 mM in the final composition.

In some embodiments, the aqueous assay sample comprises NaCl, CaCl₂, and a buffering agent. In some embodiments, the NaCl is about 10 mM to about 500 mM, the CaCl₂ is about 0.1 mM to about 20 mM, and the buffering agent is about 5 mM to about 200 mM in the aqueous assay sample. In some embodiments, the NaCl is about 25 mM to about 400 mM, the CaCl₂ is about 0.2 mM to about 10 mM, and the buffering agent is about 10 mM to about 100 mM in the aqueous assay sample. In some embodiments, the NaCl is about 50 mM to about 300 mM, the CaCl₂ is about 0.5 mM to about 5.0 mM, and the buffering agent is about 20 mM to about 8 mM in the aqueous assay sample. In some embodiments, the NaCl is about 75 mM to about 250 mM, the CaCl₂ is about 0.7 to about 3.0 mM, and the buffering agent is about 30 mM to about 70 mM in the aqueous assay sample. In some embodiments, the NaCl is about 100 mM to about 200 mM, the CaCl₂ is about 1.0 to about 2.0 mM, and the buffering agent is about 40 mM to about 60 mM in the aqueous assay sample. In some embodiments, the NaCl is about 150 mM, the CaCl₂ is about 0.3 mM, and the buffering agent is about 45 mM to about 55 mM in the aqueous assay sample. In some embodiments, the buffering agent is Bis-Tris. In some embodiments, the buffering agent is Tris. One of skill in the art would recognize that salts and buffers are commonly found in a protein preparation, and thus the above percentages are provided by example only.

In some embodiments, the pH of the aqueous assay sample is adjusted to maximize the fluorescence intensity of the 4Mu. In some embodiments, the pH of the aqueous assay sample is adjusted to stabilize one or more components of the aqueous assay sample and/or the organic solvent. In some embodiments, a slightly acidic to neutral pH (e.g., about 5.0 to about 7.0) minimizes degradation of components in the aqueous assay sample. In some embodiments, a slightly acidic to neutral pH (e.g., about 5.0 to about 7.0) minimizes aggregation of the therapeutic protein. In some embodiments, a slightly acidic to neutral pH (e.g., about 5.0 to about 7.0) minimizes autohydrolysis of the 4-methylumbelliferyl substrate (4Mu).

In some embodiments, the aqueous assay sample has an acidic pH. In some embodiments, the aqueous assay sample has a pH of 5.0 to 7.0. In some embodiments, the aqueous assay sample has a pH of about 5.0, about 5.1, about 5.2, about 5.3, about 5.4, about 5.5, about 5.6, about 5.7, about 5.8, about 5.9, about 6.0, about 6.1, about 6.2, about 6.3, about 6.4, about 6.5, about 6.6, about 6.7, about 6.8, about 6.9, or about 7.0.

In some embodiments, the composition of the present disclosure comprises an aqueous assay sample as described herein, and an organic solvent. As used herein, “organic solvent” refers a carbon-based substance that can be used to dissolve one or more solutes. Examples of organic solvents include, but are not limited to, hydrocarbons including, e.g., aliphatic hydrocarbons, cyclic hydrocarbons, aromatic hydrocarbons, and halogenated hydrocarbons; ketones; amines; esters; alcohols; aldehydes; ethers; nitriles; sulfoxides; and the like. In some embodiments, the organic solvent is capable of solubilizing the 4-methylumbelliferyl substrate (4Mu).

In some embodiments, the organic solvent comprises an alcohol, a sulfoxide, a nitrile, or combination thereof. In some embodiments, the organic solvent is dimethyl sulfoxide (DMSO). In some embodiments, the organic solvent comprises acetonitrile (ACN). In some embodiments, the organic solvent comprises an alcohol. In some embodiments, the organic solvent is a C₁-C₆ alcohol. In some embodiments, the C₁-C₆ alcohol is methanol, ethanol, propanol, iso-propanol, butanol, sec-butanol, tert-butanol, pentanol, or hexanol. In some embodiments, the organic solvent is methanol, ethanol, propanol, iso-propanol, butanol, sec-butanol, tert-butanol, or combination thereof. In some embodiments, the organic solvent comprises a mixture of acetonitrile and an alcohol. In some embodiments, the organic solvent comprises a mixture of acetonitrile and iso-propanol. In some embodiments, the acetonitrile and iso-propanol are mixed at a ratio of about 5:1, about 4:1, about 3:1, about 2:1 or about 1:1.

Various concentrations of 4Mu can be used in the compositions and methods described herein. Generally, the amount of 4Mu should be minimized to minimize the effect of autohydrolysis. In some embodiments, the 4Mu in the organic solvent is about 1 µM to about 1 mM, or about 10 µM to about 500 µM, or about 20 µM to about 200 µM, or about 50 µM to about 150 µM, or about 75 µM to about 125 µM, or about 100 µM.

In some embodiments, the composition comprises the aqueous assay sample comprising the protein preparation as described herein; and the organic solvent comprising 4-methylumbelliferyl carboxylate ester as described herein. In some embodiments, the composition does not comprise an equal volume of the aqueous assay sample and the organic solvent. In some embodiments, the amount of organic solvent in the composition is less than the amount of aqueous assay buffer in the composition, in order to minimize potentially adverse effects of the organic solvent on the protein preparation, in particular the therapeutic protein. For example, if the amount of organic solvent is too high, the therapeutic protein may aggregate. In some embodiments, the aqueous assay sample is about 70% to about 99.9% by volume of the composition, and the organic solvent is about 0.1% to about 30% by volume of the composition. In some embodiments, the aqueous assay sample is about 70% to about 99.5% by volume of the composition, and the organic solvent is about 0.5% to about 30% by volume of the composition. In some embodiments, the aqueous assay sample is about 70% to about 99% by volume of the composition, and the organic solvent is about 1% to about 30% by volume of the composition. In some embodiments, the aqueous assay sample is about 75% to about 99% by volume of the composition, and the organic solvent is about 1% to about 25% by volume of the composition. In some embodiments, the aqueous assay sample is about 80% to about 98% by volume of the composition, and the organic solvent is about 2% to about 20% by volume of the composition. In some embodiments, the aqueous assay sample is about 90% to about 98% by volume of the composition, and the organic solvent is about 2% to about 10% by volume of the composition. In some embodiments, the aqueous assay sample is about 95% to about 98% by volume of the composition, and the organic solvent is about 2% to about 5% by volume of the composition.

In some embodiments, the aqueous assay sample is about 80%, about 81%, about 82%, about 83%, about 84%, about 85%, about 85%, about 86%, about 87%, about 88%, about 89%, about 90%, about 91%, about 92%, about 93%, about 94%, about 95%, about 96%, about 97%, about 98%, about 99%, about 99.1%, about 99.2%, about 99.3%, about 99.4%, about 99.5%, about 99.6%, about 99.7%, about 99.8%, or about 99.9% by volume of the composition. In some embodiments, the protein preparation is about 70% to about 85%, about 75% to about 85%, or about 80% to about 85% by volume of the composition, and the non-protein preparation components of the aqueous assay sample, e.g., the buffering agent and/or salt, comprise about 15% to about 30%, about 15% to about 25%, about 15% to about 20% by volume of the composition. In some embodiments, the organic solvent is about 0.1%, about 0.2%, about 0.3%, about 0.4%, about 0.5%, about 0.6%, about 0.7%, about 0.8%, about 0.9%, about 1%, about 2%, about 3%, about 4%, about 5%, about 6%, about 7%, about 8%, about 9%, about 10%, about 11%, about 12%, about 13%, about 14%, about 15%, about 16%, about 17%, about 18%, about 19%, about 20%, about 25%, about 30%, about 35%, about 40%, about 45%, or about 50% by volume of the composition. One of skill in the art would recognize that salts and buffers are commonly found in a protein preparation, and thus the above percentages are used by example only.

In some embodiments, the composition further comprises a lipase inhibitor. In some embodiments, the lipase inhibitor reduces or abolishes lipolytic activity in the composition by inactivating a lipase. In some embodiments, the lipase inhibitor is included in the composition to provide a negative control for detection of lipolytic activity, i.e., a composition comprising a lipase inhibitor is not expected to have lipolytic activity. In some embodiments, the lipase inhibitor is added to the composition after detecting lipolytic activity, e.g., by measuring fluorescence of 4Mu. In some embodiments, the lipase inhibitor is in the aqueous assay sample. In some embodiments, the lipase inhibitor is water soluble. In some embodiments, the lipase inhibitor is in the organic solvent. In some embodiments, the lipase inhibitor is not water soluble.

In some embodiments, the lipase inhibitor is at a concentration sufficient to reduce or abolish lipolytic activity in the composition. In some embodiments, the lipase inhibitor is at a concentration sufficient to reduce lipolytic activity in the composition by about 50%, about 60%, about 70%, about 80%, about 90%, about 95%, about 99% or about 100% in the composition. In some embodiments, the lipase inhibitor is about 1 µM to about 50 µM in the composition. In some embodiments, the lipase inhibitor is about 2 µM to about 40 µM in the composition. In some embodiments, the lipase inhibitor is about 3 µM to about 35 µM in the composition. In some embodiments, the lipase inhibitor is about 4 µM to about 30 µM in the composition. In some embodiments, the lipase inhibitor is about 5 µM to about 25 µM in the composition. In some embodiments, the lipase inhibitor is about 1 µM, about 2 µM, about 3 µM, about 4 µM, about 5 µM, about 6 µM, about 7 µM, about 8 µM, about 9 µM, about 10 µM, about 11 µM, about 12 µM, about 13 µM, about 14 µM, about 15 µM, about 16 µM, about 17 µM, about 17 µM, about 18 µM, about 19 µM, about 20 µM, about 21 µM, about 22 µM, about 23 µM, about 24 µM, about 25 µM, about 30 µM, about 35 µM, about 40 µM, about 45 µM, or about 50 µM in the composition.

In some embodiments, the lipase inhibitor is (S)-2-formylamino-4-methyl-pentanoic acid (S)-1-[[(2S,3S)-3-hexyl-4-oxo-2-oxetanyl]methyl]-dodecyl ester (orlistat). In some embodiments, the lipase inhibitor is an alkaloid, e.g., caffeine, theophylline, and theobromine. In some embodiments, the lipase inhibitor is a carotenoid such as, e.g., fucoxanthin. In some embodiments, the lipase inhibitor is a glycoside, e.g., acteoside, kaempferol-3-O-rutinoside, rutin, kaempferol, quercetin, and luteolin. In some embodiments, the lipase inhibitor is a polyphenol, e.g., galangin, hesperidin, licohalcone A, CT-II, 7-phloroeckol, and isoliquiritigenin. In some embodiments, the lipase inhibitor is a saponin, e.g., sessiloside and chiianoside. In some embodiments, the lipase inhibitor is a terpene, e.g., crocin and crocetin. In some embodiments, the lipase inhibitor is derived from bacteria, e.g., lipstatin, valilactone, percyquinnin, panclicin, ebelactone, vibralactone, and esterastin. In some embodiments, the lipase inhibitor is a synthetic lipase inhibitor, e.g., synthetic analogs of natural fats. Lipase inhibitors are reviewed in Lunagariya yet al., EXCLI J 13: 897-921 (2014).

In some embodiments, the disclosure provides a composition comprising: (a) about 90% to about 99.9% (vol/vol) of an aqueous assay sample comprising (i) a purified protein preparation comprising a protein and a lipid; (ii) a buffering agent; (iii) about 1.0 mM to about 2.0 mM calcium chloride; and (iv) about 100 mM to about 200 mM sodium chloride; and (b) about 10% to about 0.1% (vol/vol) of an organic solvent selected from methanol, ethanol, propanol, iso-propanol, butanol, sec-butanol, iso-butanol, tert-butanol, dimethyl sulfoxide (DMSO), acetonitrile, or combinations thereof, further comprising of 4-methylumbelliferyl carboxylate ester; wherein the aqueous assay sample has a pH of 5.0 to 7.0.

In some embodiments, the disclosure provides a composition comprising: (a) about 90% to about 99.9% (vol/vol) of an aqueous assay sample comprising (i) a purified protein preparation comprising a protein and a polysorbate surfactant; (ii) a buffering agent; (iii) about 1.0 mM to about 2.0 mM calcium chloride; and (iv) about 100 mM to about 200 mM sodium chloride; and (b) about 10% to about 0.1% (vol/vol) of an organic solvent selected from methanol, ethanol, propanol, iso-propanol, butanol, sec-butanol, iso-butanol, tert-butanol, dimethyl sulfoxide (DMSO), acetonitrile, or combinations thereof, further comprising a 4-methylumbelliferyl carboxylate ester; wherein the aqueous assay sample has a pH of 5.0 to 7.0.

In some embodiments, the present disclosure provides a composition comprising: (a) about 90% to about 99.9% (vol/vol) of an aqueous assay sample comprising (i) a partially purified protein preparation comprising a protein and a lipid; (ii) a buffering agent; (iii) about 1.0 mM to about 2.0 mM calcium chloride; and (iv) about 100 mM to about 200 mM sodium chloride; and (b) about 10% to about 0.1% (vol/vol) of an organic solvent selected from methanol, ethanol, propanol, iso-propanol, butanol, sec-butanol, iso-butanol, tert-butanol, dimethyl sulfoxide (DMSO), acetonitrile, or combinations thereof, further comprising a of 4-methylumbelliferyl carboxylate ester; wherein the aqueous assay sample has a pH of 5.0 to 7.0.

In some embodiments, the present disclosure provides a composition comprising: (a) about 90% to about 99.9% (vol/vol) of an aqueous assay sample comprising (i) a cell culture supernatant comprising a protein and a lipid; (ii) a buffering agent; (iii) about 1.0 mM to about 2.0 mM calcium chloride; and (iv) about 100 mM to about 200 mM sodium chloride; and (b) about 10% to about 0.1% (vol/vol) of an organic solvent selected from methanol, ethanol, propanol, iso-propanol, butanol, sec-butanol, iso-butanol, tert-butanol, dimethyl sulfoxide (DMSO), acetonitrile, or combinations thereof, further comprising 4-methylumbelliferyl carboxylate ester; wherein the aqueous assay sample has a pH of 5.0 to 7.0.

In additional embodiments, the compositions provided herein are suitable to be used in a method for detecting lipolytic activity in a protein preparation. In some embodiments, the present disclosure further provides methods of detecting lipolytic activity in an aqueous assay sample.

In some embodiments, the present disclosure provides a method of detecting lipolytic activity in an aqueous assay sample, the method comprising (a) combining the aqueous assay sample comprising a protein preparation with an organic solvent comprising of 4-methylumbelliferyl carboxylate ester; and (b) measuring the formation of oleate and 4-methylumbelliferone (4Mu) by fluorescence.

In some embodiments, the aqueous assay sample is an aqueous assay sample described herein. In some embodiments, the aqueous assay sample has a pH of 5.0 to 7.0.

In some embodiments, the aqueous assay sample further comprises a buffering agent, a salt, or both, as described herein. Examples of buffering agents and salts and concentrations thereof suitable for the present methods are also provided herein. In some embodiments, the salt is sodium chloride (NaCl), calcium chloride (CaCl₂), or combinations thereof. In some embodiments, the salt is sodium chloride and calcium chloride. In some embodiments, the sodium chloride is about 50 mM to about 400 mM in the aqueous assay sample. In some embodiments, the sodium chloride is about 100 mM to about 200 mM in the aqueous assay sample. In some embodiments, the calcium chloride is about 0.2 mM to about 10 mM in the aqueous assay sample. In some embodiments, the calcium chloride is about 1.0 mM to about 2.0 mM in the aqueous assay sample.

In some embodiments, the buffering agent has a buffering capacity at about pH 6.0. In some embodiments, the buffering agent is Tris. In some embodiments, the buffering agent is Bis-Tris. In some embodiments, the buffering agent is about 2 mM to about 200 mM in the aqueous assay sample. In some embodiments, the buffering agent is about 10 mM to about 100 mM in the aqueous assay sample. In some embodiments, the buffering agent is about 40 mM to about 60 mM in the aqueous assay sample. In some embodiments, the buffering agent is about 45 mM to about 55 mM in the aqueous assay sample.

In some embodiments, the organic solvent is an organic solvent described herein. In some embodiments, the organic solvent is an alcohol, a sulfoxide, a nitrile, or combination thereof. In some embodiments, the organic solvent is dimethyl sulfoxide (DMSO). In some embodiments, the organic solvent comprises acetonitrile. In some embodiments, the organic solvent comprises an alcohol. In some embodiments, the organic solvent is a C₁-C₆ alcohol. In some embodiments, the organic solvent is methanol, ethanol, propanol, iso-propanol, butanol, sec-butanol, tert-butanol, or combination thereof. In some embodiments, the organic solvent comprises a mixture of acetonitrile and isopropanol. In some embodiments, the acetonitrile and isopropanol are mixed at a ratio of about 3:1.

In some embodiments, the organic solvent of 4-methylumbelliferyl carboxylate ester. The structure of various 4-methylumbelliferyl carboxylate esters is provided herein. In some embodiments, 4-methylumbelliferyl carboxylate ester hydrolyzes to form carboxylate ester and 4-methylumbelliferone (4Mu). The structure of 4Mu is provided herein. In some embodiments, 4Mu is fluorescent. In some embodiments, 4Mu fluorescence is measured at about 330 nm excitation and 495 mm emission.

In some embodiments, the method comprises measuring fluorescence for up to 24 hours. In some embodiments, the fluorescence is measured for about 24 hours to about 400 hours. In some embodiments, the fluorescence is measured for greater than about 24 hours. In some embodiments, the fluorescence is measured for greater than about 100 hours. In some embodiments, the fluorescence is measured for greater than about 300 hours. It should be understood that the fluorescence measurement is not necessarily a continuous measurement, and that the fluorescence can be measured at predetermined time points. In some embodiments, the fluorescence is measured at selected time points between about 12 hours to about 400 hours. In some embodiments, the fluorescence is measured at a time point of about 24 hours, about 48 hours, about 72 hours, about 96 hours, about 120 hours, about 144 hours, about 168 hours, about 192 hours, about 216 hours, about 240 hours, about 264 hours, about 288 hours, about 312 hours, about 336 hours, about 360 hours, about 384 hours, or about 400 hours. The period of time for which fluorescence is measured can be chosen on the level of lipase activity in the protein preparation. For example, low levels of lipolytic activity may require a longer period of detection due to slower hydrolysis of a 4-methylumbelliferyl carboxylate ester.

In some embodiments, the aqueous assay sample and the organic solvent are combined at a ratio of about 70:30 to about 99:1. In some embodiments, the aqueous assay sample and the organic solvent are combined at a ratio of about 75:25 to about 99:1. In some embodiments, the aqueous assay sample and the organic solvent are combined at a ratio of about 80:20 to about 98:2. In some embodiments, the aqueous assay sample and the organic solvent are combined at a ratio of about 85:15 to about 98:2. In some embodiments, the aqueous assay sample and the organic solvent are combined at a ratio of about 90:10 to about 98:2. In some embodiments, the aqueous assay sample and the organic solvent are combined at a ratio of about 95:5 to about 98:2.

In some embodiments, the aqueous assay sample is incubated with a lipase inhibitor for about 10 minutes to about 1 hour prior to step (a), combining the aqueous assay sample comprising the protein preparation and the organic solvent. In some embodiments, the aqueous assay sample is incubated with a lipase inhibitor for about 15 minutes to about 45 minutes, for about 20 minutes to about 40 minutes, or for about 30 minutes prior to step (a). In some embodiments, incubation of the aqueous assay sample with a lipase inhibitor reduces or abolishes lipolytic activity. In some embodiments, incubation of the aqueous assay sample with a lipase inhibitor provides a negative control for the detection of lipolytic activity. In embodiments wherein the aqueous assay sample is incubated with a lipase inhibitor prior to step (a), the measured fluorescence is expected to be low, i.e., indicating low or lack of lipolytic activity. Lipase inhibitors are described herein. In some embodiments, the lipase inhibitor is (S)-2-formylamino-4-methyl-pentanoic acid (S)-1-[[(2S,3S)-3-hexyl-4-oxo-2-oxetanyl]methyl]-dodecyl ester (orlistat). In some embodiments, the lipase inhibitor is about 1 µM to about 50 µM in the composition. In some embodiments, the lipase inhibitor is about 5 µM to about 25 µM in the composition.

### Kits

In some embodiments, the disclosure provides for kits that are suitable for providing the compositions of the present invention. In some embodiments, the disclosure provides for a kit which can be used to accomplish the methods of the present invention. For example, in some embodiments, the present disclosure further provides a kit comprising, in two or more containers: (a) an organic solvent; (b) 4-methylumbelliferyl carboxylate ester; and (c) a lipase inhibitor.

Any suitable container can be used in the kits described herein. In some embodiments, the container is a vial. In some embodiments, the container is a bottle. In some embodiments, each container is a compartment of a multi-compartment container. In some embodiments, the organic solvent and the 4-methylumbelliferyl carboxylate ester are in a first container, and the lipase inhibitor is in a second container. In some embodiments, the organic solvent and the lipase inhibitor are in a first container, and the 4-methylumbelliferyl carboxylate ester is in a second container. In some embodiments, the lipase inhibitor and the 4-methylumbelliferyl carboxylate ester are in a first container, and the organic solvent is in a second container. In some embodiments, the lipase inhibitor and the organic solvent are in a first container, and the 4-methylumbelliferyl carboxylate ester and the organic solvent are in a second container. In some embodiments, the 4-methylumbelliferyl carboxylate ester is provided as a solid, e.g., a powder. In some embodiments, the 4-methylumbelliferyl carboxylate ester is provided in solution, e.g., in the organic solvent. In some embodiments, the lipase inhibitor is provided as a solid, e.g., a powder such as a lyophilized powder. In some embodiments, the lipase inhibitor is provided in solution, e.g., in the organic solvent. In any of the above embodiments, the (a) an organic solvent; (b) 4-methylumbelliferyl carboxylate ester; and/or (c) a lipase inhibitor can be include in their respective containers to receive a predetermined specific amount of protein preparation, wherein the amount of each component is sufficient to practice the method of determining lipolytic activity described herein. In some embodiments, the kit further comprises instructions for utilizing the kit to determine lipolytic activity as in the methods described herein.

In some embodiments, the kit further comprises a buffering agent, a salt, or both. Suitable buffering agents and salts are described herein. In some embodiments, a user of the kit provides a protein preparation for use with the kit. In some embodiments, the protein preparation of the user is in a buffer unsuitable for use with the kit, e.g., a buffer that promotes auto-hydrolysis of the 4-methylumbelliferyl carboxylate ester and/or degradation of the lipase inhibitor. In some embodiments, the kit provides a buffer exchange column. In some embodiments, the buffer exchange column exchanges the buffer of the user’s protein preparation into a buffer suitable for use with the kits provided herein. Examples of buffer exchange columns include, but are not limited to, ZEBA® columns from THERMO FISHER, PD-10®, SEPHADEX®, HIPREP®, and HITRAP® columns from GE HEALTHCARE, VIVAFLOW® and VIVASPIN® concentrators from SARTORIUS, BIO-SPIN® and ECONO® columns from BIO-RAD, and SPINOUT® columns from G-BIOSCIENCES.

The columns of the kits described herein can be used to exchange the buffer system. Columns used for this purpose are known to the skilled artisan. For example, the column can be used to exchange the buffer in a protein preparation to a buffer more suitable to practice the methods of determining lipolytic activity as described herein.

In some embodiments, the present disclosure provides a kit comprising: (a) an organic solvent comprising a 4-methylumbelliferyl substrate (4Mu); (b) a column suitable for exchanging buffer of a protein preparation; and (c) a lipase inhibitor.

Suitable organic solvents for kits of the present disclosure include organic solvents described herein. In some embodiments, the organic solvent is an alcohol, a sulfoxide, a nitrile, or combination thereof. In some embodiments, the organic solvent is dimethyl sulfoxide (DMSO). In some embodiments, the organic solvent comprises acetonitrile. In some embodiments, the organic solvent comprises an alcohol. In some embodiments, the organic solvent is a C₁-C₆ alcohol. In some embodiments, the organic solvent is methanol, ethanol, propanol, iso-propanol, butanol, sec-butanol, iso-butanol, tert-butanol, or combinations thereof. In some embodiments, the organic solvent comprises a mixture of acetonitrile and isopropyl alcohol.

Suitable lipase inhibitors for kits of the present disclosure include lipase inhibitors described herein. In some embodiments, the lipase inhibitor is (S)-2-formylamino-4-methyl-pentanoic acid (S)-1-[[(2S,3S)-3-hexyl-4-oxo-2-oxetanyl]methyl]-dodecyl ester (orlistat). In some embodiments, the lipase inhibitor is used as a control when practicing the method of determining lipolytic activity as described herein.

Suitable salts for kits of the present disclosure include salts described herein. In some embodiments, the salt is sodium chloride, calcium chloride or combinations thereof. In some embodiments, the salt is sodium chloride and calcium chloride.

Suitable buffering agents for kits of the present disclosure include buffering agents described herein. In some embodiments, the buffering agent is Tris. In some embodiments, the buffering agent is Bis-Tris.

In some embodiments, the kit further comprises instructions for performing an assay to determine lipolytic activity. In some embodiments, the assay comprises a method described herein.

All references cited herein, including patents, patent applications, papers, textbooks and the like, and the references cited therein, to the extent that they are not already, are hereby incorporated herein by reference in their entirety.

### Definitions

It will be understood that descriptions herein are exemplary and explanatory only and are not restrictive of the invention as claimed. In this application, the use of the singular includes the plural unless specifically stated otherwise.

The section headings used herein are for organizational purposes only and are not to be construed as limiting the subject matter described. All documents, or portions of documents, cited in this application, including but not limited to patents, patent applications, articles, books, and treatises, are hereby expressly incorporated by reference in their entirety for any purpose. As utilized in accordance with the present disclosure, the following terms, unless otherwise indicated, shall be understood to have the following meanings:

As used herein, “a” or “an” may mean one or more. As used herein, when used in conjunction with the word “comprising,” the words “a” or “an” may mean one or more than one. As used herein, “another” or “a further” may mean at least a second or more.

Throughout this application, the term “about” is used to indicate that a value includes the inherent variation of error for the method/device being employed to determine the value, or the variation that exists among the study subjects. Typically, the term “about” is meant to encompass approximately or less than 1%, 2%, 3%, 4%, 5%, 6%, 7%, 8%, 9%, 10%, 11%, 12%, 13%, 14%, 15%, 16%, 17%, 18%, 19% or 20% or higher variability, depending on the situation. In some embodiments, one of skill in the art will understand the level of variability indicated by the term “about,” due to the context in which it is used herein. It should also be understood that use of the term “about” also includes the specifically recited value.

The use of the term “or” in the claims is used to mean “and/or,” unless explicitly indicated to refer only to alternatives or the alternatives are mutually exclusive, although the disclosure supports a definition that refers to only alternatives and “and/or.”

As used herein, the terms “comprising” (and any variant or form of comprising, such as “comprise” and “comprises”), “having” (and any variant or form of having, such as “have” and “has”), “including” (and any variant or form of including, such as “includes” and “include”) or “containing” (and any variant or form of containing, such as “contains” and “contain”) are inclusive or open-ended and do not exclude additional, unrecited elements or method steps. It is contemplated that any embodiment discussed in this specification can be implemented with respect to any method, composition, and/or kit of the present disclosure. Furthermore, compositions of the present disclosure can be used to achieve methods and kits of the present disclosure.

The use of the term “for example” and its corresponding abbreviation “e.g.” (whether italicized or not) means that the specific terms recited are representative examples and embodiments of the disclosure that are not intended to be limited to the specific examples referenced or cited unless explicitly stated otherwise.

As used herein, “between” is a range inclusive of the ends of the range. For example, a number between x and y explicitly includes the numbers x and y, and any numbers that fall within x and y.

As used herein, “protein,” “peptide,” or “polypeptide” refer to a polymeric form of amino acids, which can be any length. Proteins can include, e.g., antibodies, structural proteins, enzymes, membrane, membrane-associated, and/or transmembrane proteins, transporters, receptors, signaling proteins, and the like. Proteins and/or peptides of the present disclosure also encompass modified proteins, e.g., conjugated to one or more non-peptide substances such as, e.g., a drug, a targeting moiety, a tag such as a visualization tag, and the like. A protein of the present disclosure can be a therapeutic protein, e.g., used in diagnosis, treatment, and/or prevention of a disease or disorder. In some embodiments, a polysorbate described herein can improve stability of the protein in a pharmaceutical formulation. In some embodiments, the therapeutic protein is an antibody. In some embodiments, the therapeutic protein is an antibody-drug conjugate. In some embodiments, a protein preparation described herein includes a protein, e.g., a therapeutic protein.

The term “isolated” means (i) free of at least some other proteins with which it would normally be found, (ii) is essentially free of other proteins from the same source, e.g., from the same species, (iii) separated from at least about 50 percent of polynucleotides, lipids, carbohydrates, or other materials with which it is associated in nature, (iv) operably associated (by covalent or noncovalent interaction) with a polypeptide with which it is not associated in nature, or (v) does not occur in nature.

A “variant” of a polypeptide (e.g., an antigen-binding molecule) comprises an amino acid sequence wherein one or more amino acid residues are inserted into, deleted from and/or substituted into the amino acid sequence relative to another polypeptide sequence. Variants include, e.g., fusion proteins.

The term “derivative” refers to a molecule that includes a chemical modification other than an insertion, deletion, or substitution of amino acids (or nucleic acids). In certain embodiments, derivatives comprise covalent modifications, including, but not limited to, chemical bonding with polymers, lipids, or other organic or inorganic moieties. In certain embodiments, a chemically modified antigen-binding molecule can have a greater circulating half-life than an antigen-binding molecule that is not chemically modified. In some embodiments, a derivative antigen-binding molecule is covalently modified to include one or more water soluble polymer attachments, including, but not limited to, polyethylene glycol, polyoxyethylene glycol, or polypropylene glycol.

Standard techniques can be used for recombinant DNA, oligonucleotide synthesis, and tissue culture and transformation (e.g., electroporation, lipofection). Enzymatic reactions and purification techniques can be performed according to manufacturer’s specifications or as commonly accomplished in the art or as described herein. The foregoing techniques and procedures can be generally performed according to conventional methods well known in the art and as described in various general and more specific references that are cited and discussed throughout the present specification. See, e.g., Sambrook et al., Molecular Cloning: A Laboratory Manual (2d ed., Cold Spring Harbor Laboratory Press, Cold Spring Harbor, N.Y. (1989)), which is incorporated herein by reference for any purpose.

As used herein, the term “substantially” or “essentially” refers to a quantity, level, value, number, frequency, percentage, dimension, size, amount, weight or length that is about 90%, 91 %, 92%, 93%, 94%, 95%, 96%, 97%, 98%, or 99% higher compared to a reference quantity, level, value, number, frequency, percentage, dimension, size, amount, weight or length. In one embodiment, the terms “essentially the same” or “substantially the same” refer to a range of quantity, level, value, number, frequency, percentage, dimension, size, amount, weight or length that is about the same as a reference quantity, level, value, number, frequency, percentage, dimension, size, amount, weight or length.

As used herein, the terms “substantially free of′ and “essentially free of′ are used interchangeably, and when used to describe a composition, such as a cell population or culture media, refer to a composition that is free of a specified substance, such as, 95% free, 96% free, 97% free, 98% free, 99% free of the specified substance, or is undetectable as measured by conventional means. Similar meaning can be applied to the term “absence of,” where referring to the absence of a particular substance or component of a composition.

As used herein, the term “appreciable” refers to a range of quantity, level, value, number, frequency, percentage, dimension, size, amount, weight or length or an event that is readily detectable by one or more standard methods. The terms “not-appreciable” and “not appreciable” and equivalents refer to a range of quantity, level, value, number, frequency, percentage, dimension, size, amount, weight or length or an event that is not readily detectable or undetectable by standard methods. In one embodiment, an event is not appreciable if it occurs less than 5%, 4%, 3%, 2%, 1%, 0.1%, 0.001%, or less of the time.

Reference throughout this specification to “one embodiment,” “an embodiment,” “a particular embodiment,” “a related embodiment,” “a certain embodiment,” “some embodiments,” “an additional embodiment,” or “a further embodiment” or combinations thereof means that a particular feature, structure or characteristic described in connection with the embodiment is included in at least one embodiment of the present invention. Thus, the appearances of the foregoing phrases in various places throughout this specification are not necessarily all referring to the same embodiment. Furthermore, the particular features, structures, or characteristics may be combined in any suitable manner in one or more embodiments.

As used herein, the term “hydrolases” refers to the host cell proteins (HCPs) belonging to the Enzyme Commission (EC) main class of enzymes known as hydrolases (EC 3). As used herein, the term “esterases” mean the HCPs belonging to the subclass of hydrolases that act on ester bonds. As used herein, the term “Carboxylic ester hydrolases” mean HCPs belonging to the hydrolase sub-subclass of enzymes known carboxylic ester hydrolases, such as lipases and carboxylesterases. As used herein, the term “fluorogenic substrate” may optionally exclude 4-methylumbelliferyl oleate. As used herein, the term “HCP enzymatic activity” means determining the HCP enzymatic activity rate towards hydrolyzing the ester bond in the fluorogenic substrate. In an embodiment, determining the HCP enzymatic activity in a sample means determining the collective activity of all HCPs in the sample.

### EXAMPLES

### Example 1. Improved MU-Based Ester Substrates and Assay Conditions

To improve upon the lipase assays in the art, the following additional factors were investigated: (1) several MU-based ester substrates to identify the most sensitive substrate; (2) several model enzymes to demonstrate broader relevance; (3) several mAbs covering different formats and production hosts to ensure applicability to diverse products; (4) multiple buffer matrices spanning the ranges commonly used in protein formulations to identify potential assay interference; (5) different sample matrices, including purification in-process pools and purified materials with different formulations, to evaluate applicability towards bioprocess and formulation samples; (6) assay performance to characterize specificity, precision, limit of detection (LOD) and limit of quantitation (LOQ); (7) several purification schemes to differentiate their corresponding risks of polysorbate degradation; (8) FFA and polysorbate levels to determine correlation between esterase activity and polysorbate degradation; and (9) microplate format for higher throughput and faster assay turnaround time. Through these investigations, we developed a rapid (<3 hours in total turnaround time), high-throughput, plate-based assay that can be used to assess the risk of polysorbate degradation by detecting esterase activity towards the 4-methylumbelliferyl caprylate (MU-C8) fluorogenic substrate.

### 1. Materials and Methods

### 1.1. Materials

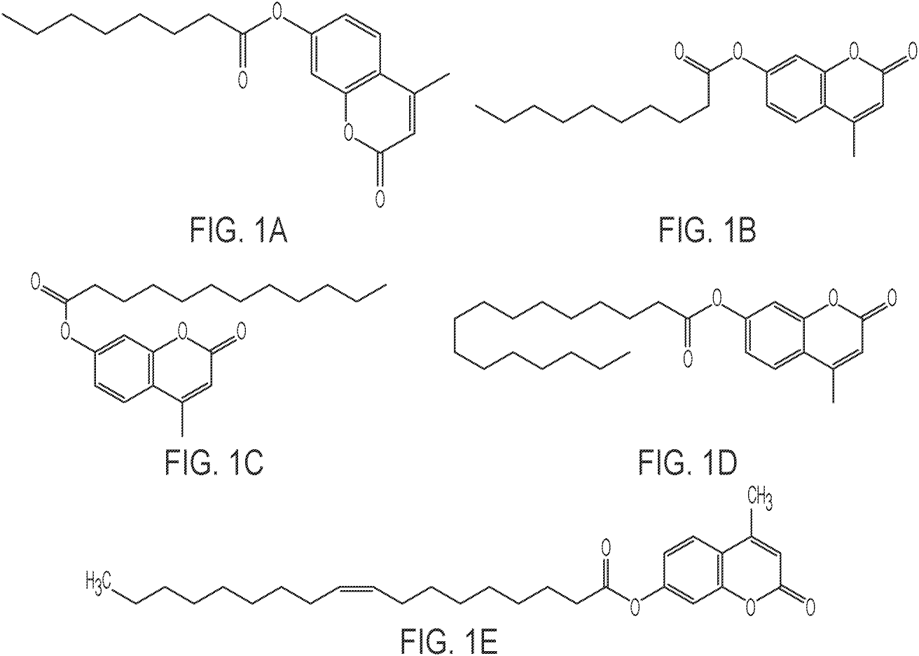

Reagents used include tris base and tris chloride (Sigma Aldrich), Triton X-100 (US Biological), gum arabic (Acros Organics), 4-methylumbelliferone (MU) and umbelliferone reaction products (Sigma Aldrich, >98% purity), orlistat (Sigma, >98% purity), and dimethyl sulfoxide (DMSO). The 4-methylumbelliferyl carboxylate substrates were purchased or custom synthesized and solubilized in DMSO prior to use. 4-methylumbelliferyl caprylate (MU-C8, Research Organics, 99% purity), 4-methylumbelliferyl decanoate (MU-C10, Santa Cruz Biotechnology, 98% purity), 4-methylumbelliferyl dodecanoate (MU-C12, Hande Sciences, >99% purity), 4-methylumbelliferyl palmitate (MU-C16, Biosynth, >99% purity), and 4-methylumbelliferyl oleate (MU-C18:1, Chemodex, >95% purity) were used and chemical structures are shown in FIG. 1. The model enzymes used were Pseudomonas cepacia lipase (PCL, Sigma Aldrich, 35 U/mg), lysosomal phospholipase A2 (LPLA2, produced in house) and phospholipase B-like 2 (PLBL2, produced in house). The seven monoclonal antibodies (mAb 1-mAb 7) used were produced in house. These mAbs were selected to cover a range of products, including different molecule formats and production hosts: mAbs 1 and 5-7 were IgG1s; mAbs 2-4 were IgG4s, including one bi-specific antibody; mAb 3 was produced by the bacterial host Escherichia coli (E. coli), whereas the other mAbs were produced by mammalian hosts of CHO lineage. All purified protein (i.e., mAb) samples tested were from ultrafiltration diafiltration (UFDF) pools unless otherwise specified. To generate the UFDF pools, CHO or E. coli harvests were purified through chromatography steps to the UFDF stage, resulting in final mAb concentrations of 57-225 mg/ml. All plates (96 well, Catalog #3882, Corning) were read using SpectraMax M2/M2e (Molecular Devices) or Synergy Neo2 (Biotek) plate readers.

### 1.2. Esterase Activity Assay

The esterase activity assay monitors the conversion of the non-fluorescent substrates, 4-methylumbelliferyl fatty acid esters, to a fluorescent product, MU, through the cleavage of the ester bond. The reaction mixture contained 80 µl of reaction buffer (150 mM tris chloride pH 8.0, 0.25% (w/v) Triton X-100 and 0.125% (w/v) gum arabic), 10 µl substrate at 1 mM final concentration, and 10 µl model enzyme solution or protein samples. The concentration of each buffer component was selected based on in-house assay development studies (data not shown). A 50 mM tris chloride running buffer at pH 8.0 with 0.01% gum arabic was previously used to determine activity rates in a lipase and esterase activity assay (Nalder TD, et al., Biochimie. 2016;128-129:127-132). Triton X-100 was shown to enhance hydrolysis of bis(4-methylumbelliferyl)phosphate (Jones CS, et al., Biochim Biophys Acta. 1982;71(3):261-268.).

Model enzymes were diluted in water to the desired concentrations. Protein samples consisted of purification in-process pool samples or purified protein samples from UFDF pools. The protein samples were assayed neat to obtain sufficient fluorescence signal or diluted by 2-4 fold when rates observed for neat samples were found to be high (>10 µM MU/h). The reaction was set up in 96-well plates and the increase of fluorescence signal (using excitation and emission wavelengths of 355 nm and 460 nm, respectively) was monitored every 10 minutes by incubating the reaction plates for two hours at 37° C. in a plate reader. Rate of production of MU, representing the total hydrolytic activity (from enzymatic and non-enzymatic hydrolysis), was derived from the slope of the fluorescence time course from 30 minutes to 120 minutes, and is denoted by the reaction raw rate (k_{raw}).

An enzyme blank reaction (i.e., negative control) was required for each sample, with identical buffer matrix to the sample but omitting the enzyme (or protein from the protein solutions). This blank reaction measured non-enzymatic hydrolysis of the substrate, which was subtracted from the total hydrolytic activity to derive the enzymatic activity of the sample. Enzyme blank reaction rate represents the non-enzymatic hydrolysis rate of the substrate in a specific sample matrix (kₙₒₙ-_{enzymatic} _{hydrolysis}).

The enzymatic hydrolysis rate is referred to herein as the esterase activity rate. For a given sample, the esterase activity rate was determined by subtracting the non-enzymatic hydrolysis rate from the reaction raw rate, and converting the fluorescence signal from relative fluorescence units per hour (RFU/h) to µM of MU produced per hour (µM MU/h). This conversion was supported by running a standard curve of free MU product on the same plate. Assay measurements were averaged across replicates and error bars represent one standard deviation (±1 SD) from the mean. The calculation for the esterase activity rate is described by equation 1:

$$E s t e r a s e A c t i v i t y R a t e = \frac{{k}_{r a w} - {k}_{n o n - e n z y m a t i c h y d r o l y s i s}}{α}$$

where k_{raw} = reaction raw rate of sample in RFU/h,
k_{non-enzymatic} _{hydrolysis} = reaction rate of enzyme blank in RFU/h,
α = calculated conversion factor of fluorescence signal to concentration of MU in RFU/µM MU.

### 1.3. Substrate Specificity

MU substrates used in the substrate specificity experiments are provided in FIG. 1. PCL, LPLA2, PLBL2, mAb 1, mAb 2, and mAb 3 were tested with five 4-methylumbelliferyl carboxylate substrates with varying chain lengths (MU-CX where X = number of carbons in the carboxylate group). Substrates were dissolved in DMSO to a final substrate concentration of 0.5 mM, which was the only modification made to the assay setup described earlier. Esterase activity rates were calculated for all samples as described earlier.

### 1.4. Orlistat Inhibition

Orlistat stock solution in DMSO was freshly prepared for each inhibition reaction. Purified mAb 1 (225 mg/ml neat, 25 mg/ml during incubation), PCL (50 ng/ml) and LPLA2 (50 ng/ml) were incubated with orlistat at final orlistat incubation concentrations of 0, 0.5, 1 and 10 µM for 2-3 hours at room temperature prior to measurement by the esterase activity assay. The enzyme blank (negative control) samples were prepared by incubating mAb 1 formulation buffer or water (used for model enzyme dilution) with orlistat. The MU standard curve also contained identical concentrations of orlistat and DMSO as the test samples to support an accurate determination of the conversion factor a in the corresponding sample matrix. Rates were adjusted for background fluorescence as measured by the enzyme blank (negative control) wells. Esterase activity rates in µM MU/h were calculated for all samples as described earlier.

To investigate the effect of orlistat on PS20 degradation, affinity chromatography purified samples of mAb 2 (28 mg/mL), mAb 6 (22 mg/ml) and mAb 7 (22 mg/ml) were incubated for 5 hours at room temperature in the presence of DMSO (10% v/v) with orlistat (0.2 µM and 20 µM) or without orlistat. The samples were then spiked with PS20 (0.04% v/v) and methionine (20 mg/ml) and incubated at 25° C. for 12 days. After incubation, the samples were analyzed for PS20 hydrolytic degradation by quantifying the FFA degradant (lauric acid) as previously described in detail (Cheng Y, et al., J Pharm Sci. 2019;108(9):2880-2886).

### 1.5. pH Dependence

Esterase activities of PCL and mAb 2 at different pHs were characterized using MU-C8 as the fluorogenic substrate. The 150 mM tris chloride based running buffer was adjusted to allow for the testing of an expanded pH range of 7-9. 150 mM sodium acetate buffer was utilized for assaying at pH 4-6. The pH of each buffer was measured prior to use to verify that the buffer was prepared to within 0.1 units of the targeted pH. To confirm that the pH drift during the course of the assay was minimal, the pH of each sample and control well was measured immediately after performing the esterase activity assay by an Apix-pH robot (AB Controls). The measured pH values were utilized to plot the pH dependence profiles.

### 1.6. Sample Matrix Interference

The impact of sample matrix (i.e., buffer composition) on non-enzymatic hydrolysis rates and fluorescent intensity of MU was assessed using MU-C8 as the fluorogenic substrate. Sodium acetate pH 5.5 (20 and 50 mM), tris acetate pH 5.5 (20 and 500 mM), histidine chloride pH 5.5 (20 and 50 mM), tris chloride pH 8.0 (20 and 200 mM), HEPES chloride pH 8.0 (20 and 200 mM), arginine chloride (500 and 1000 mM), sodium chloride (500 and 1000 mm) and sodium sulfate (500 and 600 mM) were assessed in the sample matrix study.

### 1.7. Protein and Excipient Interference

To determine potential protein interference with the esterase activity assay, 0, 20, 50, and 100 ng/ml (final concentrations) of model enzyme LPLA2 was spiked with or without 180 mg/ml of mAb 4, mixed thoroughly and immediately assayed. For the samples that were not spiked with LPLA2, water was added in place of LPLA2. In addition to this study, the fluorescence signal was measured for 0-200 mg/ml of mAb 1 mixed with the standard assay reaction buffer (no substrate) containing 0, 5 or 10 µM MU at each protein concentration level. MU concentrations were achieved by adjusting the stock concentration of MU used to spike into reaction wells and keeping the volume additions constant (10 µL). Protein interference on fluorescence was assessed after thorough mixing by comparing measured fluorescence in wells with protein (test) and without protein (blank). Finally, potential assay interference from typical formulation excipients (PS20 and PS80) was tested by spiking 0.1% PS20 or 0.1% PS80 to PCL, purified mAb 1 or purified mAb 3. Control samples (no spike) were generated by adding an equivalent volume of water in place of spiking with PS20 or PS80. All samples were mixed thoroughly and incubated for 30 minutes at room temperature prior to assaying for esterase activity using MU-C8 as the fluorogenic substrate.

### 1.8. Assay Performance Characterization

The specificity, precision (repeatability and intermediate precision), LOD and LOQ of the esterase activity assay were determined using mAb purification in-process pool samples. Three types of mAb samples were selected to cover different levels of esterase activity: (1) mAb 1 (UFDF pool sample at 220 mg/mL) represented high esterase activity; (2) mAb 6 (UFDF pool sample at 57 mg/ml) represented medium esterase activity; and (3) mAb 2 (affinity pool sample at 12 mg/ml) represented low esterase activity. First, assay specificity and precision were evaluated using six plates over two assay sessions. Assay precision in terms of repeatability was evaluated using a sample size (n) of ten per sample per plate. Assay intermediate precision was evaluated by two analysts using samples prepared from different lots of reagents and then analyzed in separate test sessions with different lots of plates. Assay specificity was evaluated by the minimum difference between MU-C8 hydrolysis rates measured in the samples and the buffer blanks (negative controls) calculated according to equation 2:

$$\begin{matrix}M i n i m u m D i f f e r e n c e i n H y d r o l y s i s R a t e s \\ = \left(M e a {n}_{s a m p l e} - S {D}_{s a m p l e}\right) - \left(M e a {n}_{b u f f e r} - S {D}_{b u f f e r}\right)\end{matrix}$$

where Meanₛₐₘₚₗₑ = Mean of MU-C8 hydrolysis rates for sample,
SDₛₐₘₚₗₑ = Standard deviation of MU-C8 hydrolysis rates for sample,
Mean_{buffer} = Mean of MU-C8 hydrolysis rates for buffer,
SD_{buffer} = Standard deviation of MU-C8 hydrolysis rates for buffer.

Next, LOD and LOQ were evaluated for three buffer systems in three plates (n=12 per plate): (i) 50 mM tris acetate at pH 5.5; (ii) 50 mM histidine chloride at pH 5.5; and (iii) 200 mM arginine chloride. The buffers were selected based on the results of the sample matrix interference studies. Assay LOD was determined as twice the one-sided 95% confidence interval for esterase activity rate (CIₑₛₜₑᵣₐₛₑ _{activity} ᵣₐₜₑ). Assay LOQ was determined as six times the one-sided 95% CIₑₛₜₑᵣₐₛₑ _{activity} ᵣₐₜₑ. The mean value of the MU-C8 hydrolysis rate for the buffer blank was not included in calculating the LOD and LOQ for the esterase activity assay because the non-enzymatic hydrolysis measured in the buffer blank was subtracted in calculating the esterase activity rate (equation 1). The one-sided 95% CIₑₛₜₑᵣₐₛₑ _{activity} ᵣₐₜₑ was calculated using equation 3:

$$95 \% C {I}_{e s t e r a s e a c t i v i t y r a t e} = \frac{1.96 \times \sqrt{{\left(S {D}_{b u f f e r}\right)}^{2} + {\left(S {D}_{s a m p l e}\right)}^{2}}}{\sqrt{n}}$$

### 1.9. Free Fatty Acid (FFA) Assay

PS80 degradation in mAb 2 purification in-process and purified UFDF pool samples was measured by quantifying FFAs before and after incubation with PS80 at 25° C. for 42 days. This method was developed and described previously by Tomlinson et al. (Mol Pharm. 2015;12(11):3805-3815). Briefly, the FFA polysorbate degradants were extracted from the samples using Oasis HLB resin, derivatized with I-pyrenyldiazomethane overnight and chromatographically analyzed using a UHPLC equipped with an Acquity PDA detector and Acquity BEH-300 C18 reverse phase column.

### 1.10. Polysorbate Content Assay

PS20 degradation in mAb 5 purified UFDF pool samples was determined by quantifying PS20 content (before and after incubation at 40° C. for 7 days) by an HPLC-ELSD method previously detailed by Hewitt et al. (2008;1215(1-2):156-160). PS80 degradation in mAb 2 purification in-process and purified UFDF pool samples was determined by quantifying PS80 content (before and after incubation at 25° C. for 42 days) using the same HPLC-ELSD method. Briefly, samples formulated with polysorbate were injected neat onto an HPLC operated in mixed mode. The hydrophobic polysorbate was retained on the Water Oasis MAX cartridge column, eluted via a step gradient, and quantified as a single peak by ELSD.

### 2. Results

### 2.1. Substrate Specificity

The three model enzymes chosen for testing MU-based fluorogenic substrates fall within the sub-subclass of carboxylic ester hydrolases (EC 3.1.1) that belong to the subclass of esterases (EC 3.1) in the main class of hydrolases (EC 3). PCL, LPLA2, and PLBL2 were selected to cover a range of enzymes that can hydrolyze the carboxylic ester bonds present in polysorbates. The purified mAbs 1-3 were selected to include different IgG subclasses (IgG1 and IgG4) and production hosts (CHO and E. coli). These model enzymes and mAbs were intended to serve as positive controls for the esterase activity assay because they were expected to degrade polysorbate. For these experiments, 0.5 mM final substrate concentration was selected due to solubility limitations for substrates with longer chain lengths such as MU-C16.

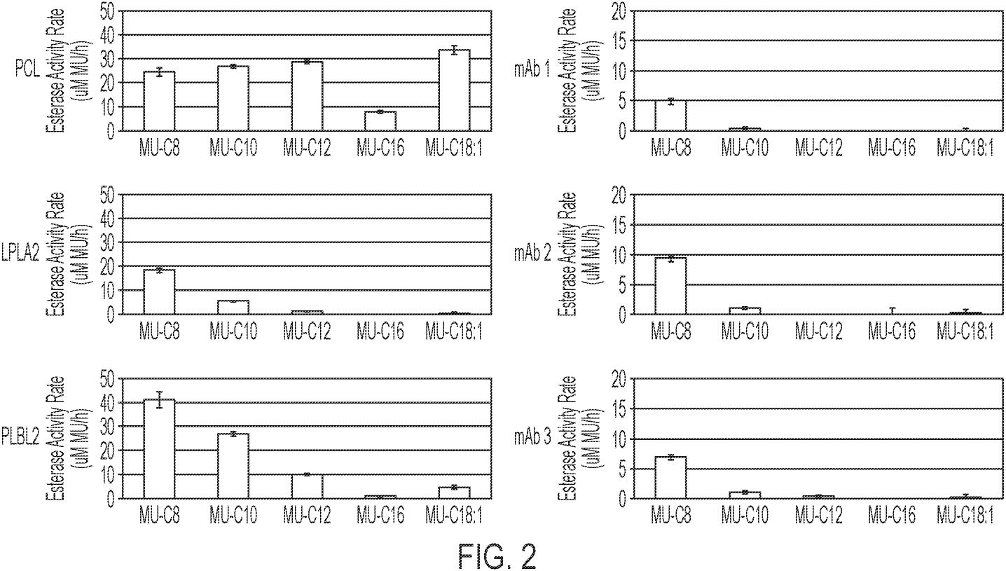

The substrate specificity experiment (FIG. 2) shows that model enzymes demonstrated activity towards MU esters for two or more of the carbon chain lengths tested (e.g., all three enzymes hydrolyzed MU-C8 and MU-C10), but protein samples demonstrated the most sensitivity to the MU-C8 substrate. For this reason, MU-C8 was chosen as the primary substrate for the esterase activity assay. Therefore, MU-C8 was the fluorogenic substrate used in all the subsequent studies hereafter.

### 2.2. Esterase Activity Assay

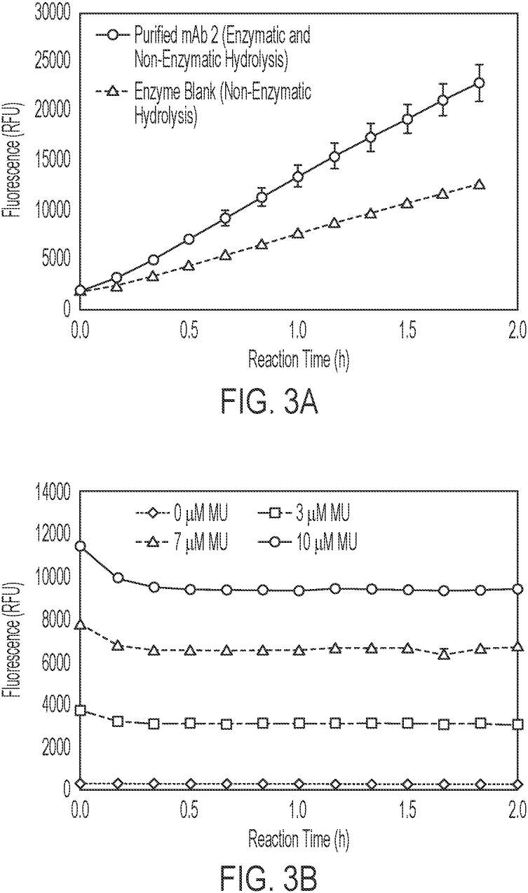

A typical fluorescence time course plot showing the reaction raw rate (k_{raw}) and reaction rate of enzyme blank (k_{non-enzymatic hydrolysis}) in the esterase activity assay (with MU-C8 substrate) is presented in FIG. 3 (left). The k_{raw} was generated in the presence of purified mAb 2 and indicates total activity (both enzymatic and non-enzymatic hydrolysis) towards the hydrolytic cleavage of the carboxylic ester bond in MU-C8. The k_{non-enzymatic} _{hydrolysis} was generated in the absence of purified mAb (i.e., negative control) and indicates background fluorescence from non-enzymatic hydrolysis of MU-C8. Time course fluorescence traces for the MU standard curve (with no MU-C8 substrate) shows a steady signal of the hydrolysis reaction product (MU) at each MU concentration across the assay duration of 30-120 minutes (FIG. 3, right). The initial change in fluorescence is due to equilibration of the assay plate from room temperature to 37° C. and the fluorescence signal stabilized after this initial time period (0-30 minutes). The k_{raw}, k_{non-enzymatic} _{hydrolysis}, and MU fluorescence are specific to the reaction conditions (e.g., pH, sample and buffer matrix). Therefore, enzyme blanks must be included as negative controls for every sample type and assay condition; the enzyme blanks account for background fluorescence in calculating the esterase activity rates.

### 2.3. Orlistat Inhibition

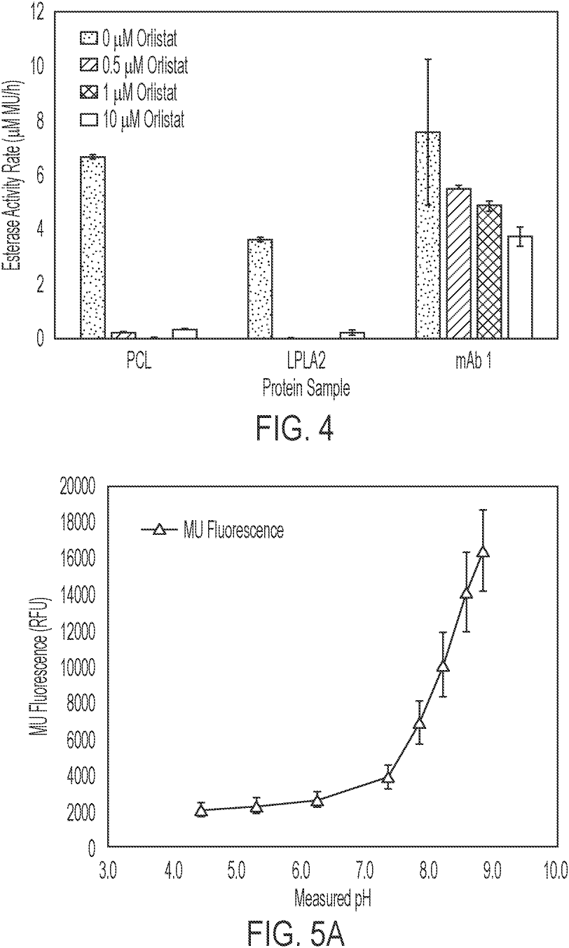

Orlistat inhibits PPL, microbial lipases, and mammalian carboxylesterases, albeit with different potencies. Therefore, orlistat was included here as a negative control for the esterase activity assay. To assess the ability of the assay to detect orlistat inhibition, mAb 1 and model enzymes PCL and LPLA2 were spiked and pre-incubated with orlistat, and then tested for esterase activity. As shown in FIG. 4, a dose-dependent reduction in esterase activity rate was observed for mAb 1 treated with orlistat compared to DMSO control (0 µM orlistat). Additionally, complete inhibition of esterase activities by the PCL and LPLA2 model enzymes were observed for the concentrations of orlistat tested.

To directly investigate the effect of Orlistat on inhibiting polysorbate degradation in mAb samples, affinity chromatography purified CHO cell culture harvests for mAb2, mAb 6, and mAb 7 were incubated with or without orlistat. Lauric acid is the primary degradant from PS20 hydrolysis, and it is therefore the most abundant and reliably quantified FFA species present in the samples. The rate of lauric acid release would indicate the rate of hydrolytic PS20 degradation in the samples. The efficacy of orlistat towards inhibiting PS20 degradation was evaluated by calculating the percent decrease in the rate of FFA release in the mAb samples with orlistat treatment relative to the same samples without orlistat treatment (Table 1). For example, complete inhibition of hydrolytic PS20 degradation by orlistat treatment would result in a 100% decrease in FFA release rate. Similar to the findings from the orlistat incubation of mAb 1 as evaluated by the esterase activity assay (FIG. 4), a dose-dependent impact of orlistat inhibition was observed for mAbs 2, 6, and 7. A decrease in the rate of PS20 degradation-as indicated by a decrease in FFA release rate-was observed for samples treated with orlistat compared to negative controls (0 µM orlistat).

TABLE 1

Orlistat incubation and corresponding impact on PS20 degradation for protein samples (mAb 2, mAb 6, and mAb 7)
Decrease in FFA Release Rate (%)^{∗}

| | mAb2 | mAb6 | mAb7 |
|---|---|---|---|
| Orlistat Concentration (µM) | | | |
| 0 (negative control) | 0 | 0 | 0 |
| 0.2 | 31 | 67 | 39 |
| 20 | 63 | 91 | 74 |

^{∗}Results reported are relative to samples without orlistat treatment (negative control). The FFA release rate was measured based on lauric acid and indicates the rate of PS20 degradation.

### 2.4. pH Dependence

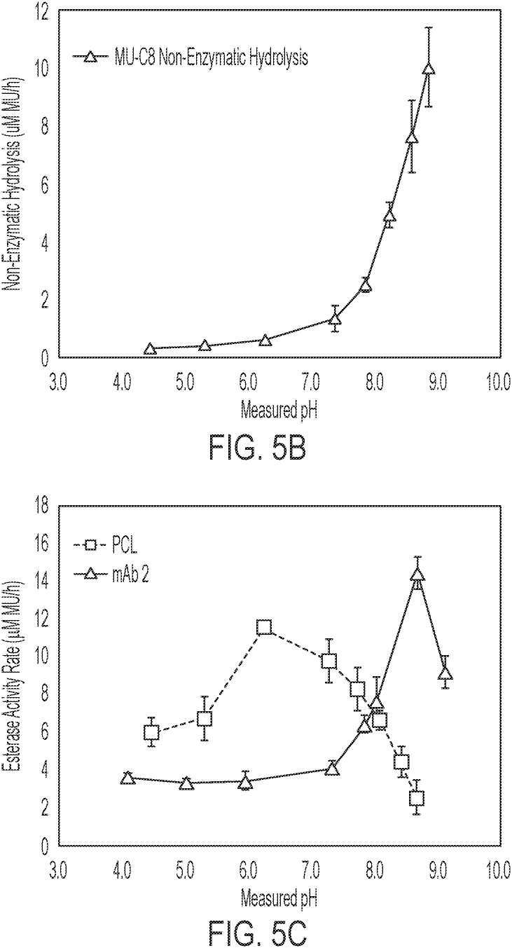

The next study investigated the pH dependence of enzymatic and non-enzymatic hydrolysis of MU-C8. For these experiments, the assay running buffer was adjusted to consist of 150 mM sodium acetate (pH 4-6) and 150 mM tris chloride (pH 7-9). The traces illustrated in FIG. 5 show the measured pH values for each sample well after the reaction was completed; the observed offsets between target pH (measured for each buffer before starting the experiment) and pH of samples measured immediately after the experiment were small. MU fluorescence and non-enzymatic hydrolysis of substrate from a nominal pH 4-9 range are shown in FIG. 5 (top, left and right). The increase in non-enzymatic hydrolysis rates with pH, measured in absence of enzyme but in the presence of substrate and reaction buffer, highlights the pH dependence of MU-C8 hydrolysis (FIG. 5, top right).

Separation of fluorescence signal from sample (with protein) and negative control (without protein) wells was carefully monitored to ensure reliable sample signal. The pH dependence profile for PCL indicates a different dependence than the trend observed with mAb 2 (FIG. 5, bottom). PCL demonstrates a lower pH optimum, while the purified mAb 2 (UFDF pool) samples demonstrate a higher pH optimum. Since the intent of the esterase activity assay is to detect hydrolytic enzyme activity in purification in-process pools and purified materials (e.g., UFDF pool, drug substance or drug product) as represented by the mAb 2 sample, pH 8.0 was selected as the default pH for this assay.

### 2.5. Buffer Matrix Interference

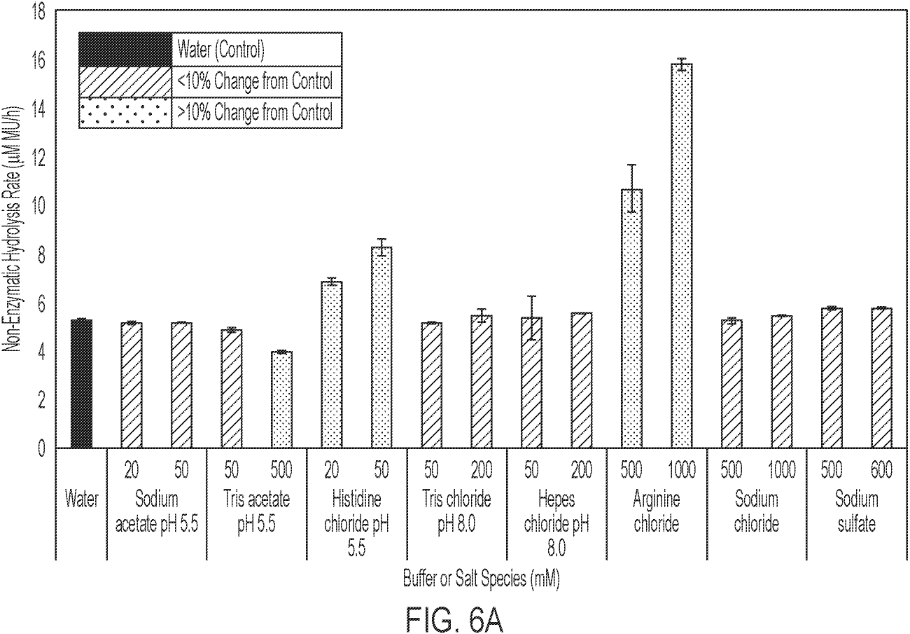

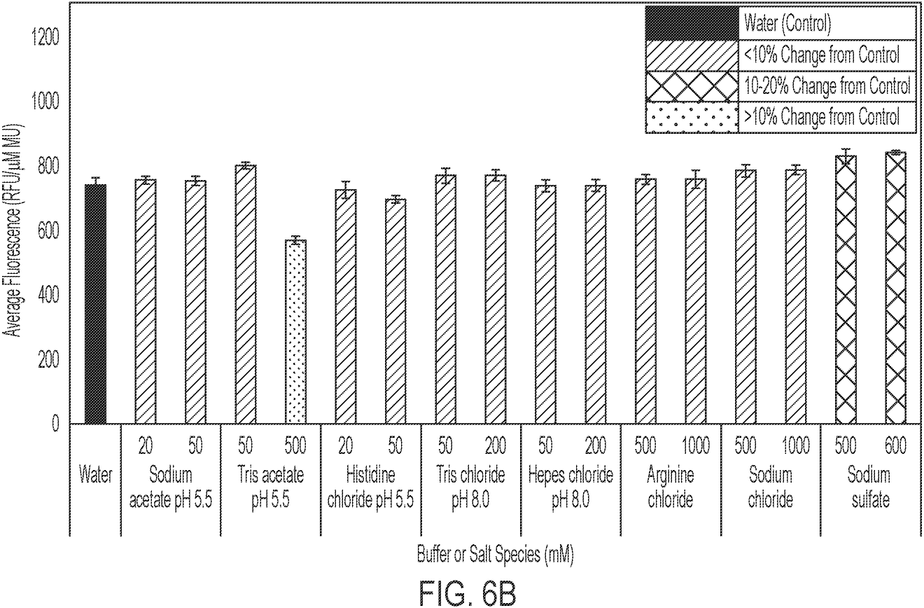

To assess the effect of buffer matrix on the assay, eight commonly used formulation buffers and salts were tested. FIG. 6 shows average MU fluorescence and substrate non-enzymatic hydrolysis with different sample matrices as compared to water (shown in black). The sample concentrations were chosen based on their relevance to purification processes and drug substance or drug product formulations. Each buffer or salt was assessed in two concentrations. Tris acetate at high concentrations (500 mM) decreased MU fluorescence by more than 20% and sodium sulfate at 500 mM and 600 mM increased MU fluorescence by 10-20% as compared to a water control. Additionally, non-enzymatic hydrolysis rates decreased with high levels of tris acetate (500 mM), and increased at the levels of histidine chloride (20 - 50 mM) and arginine chloride (500 - 1000 mM) tested. All other conditions did not result in more than a 10% change in MU fluorescence or non-enzymatic rates as compared to water control.

### 2.6. Protein and Excipient Interference

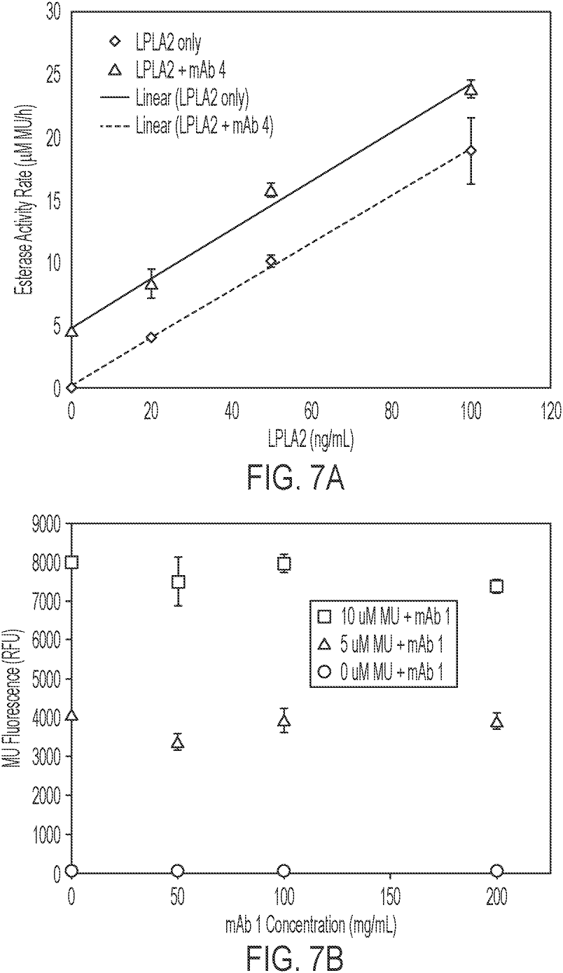

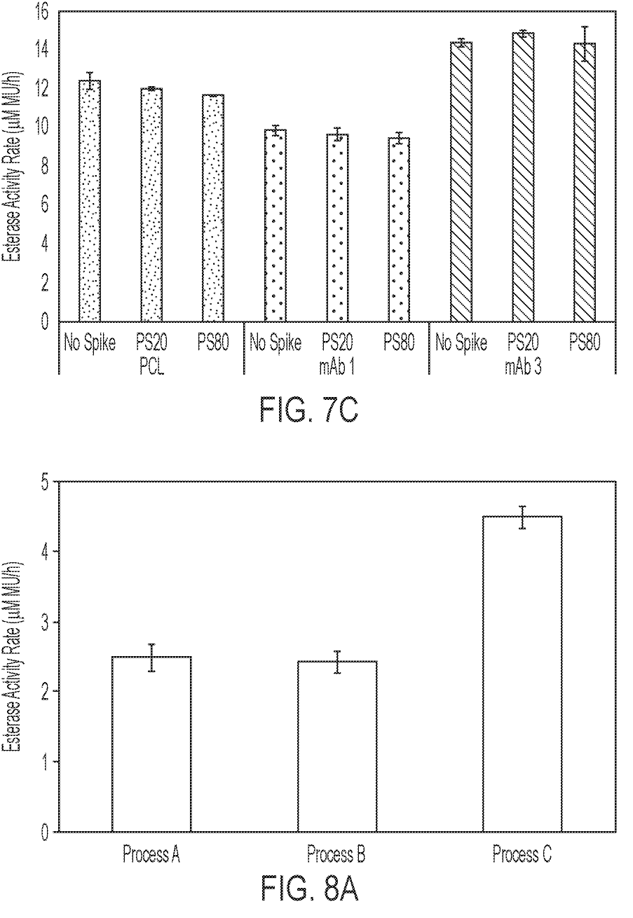

To investigate the potential impact of protein product (e.g., from in-process purification pools or purified mAbs) on the assay, three protein interference experiments were conducted. As indicated by the parallel lines in FIG. 7 (top left), the addition of 180 mg/ml of mAb 4 in up to 100 ng/ml of model enzyme LPLA2 did not change the calculated esterase activity rates. FIG. 7 (top right) also illustrates a lack of protein interference on fluorescence from adding 0, 50, 100, and 200 mg/ml of mAb 1 into 0, 5, and 10 µM MU. This further demonstrates that the presence of high concentrations of protein should not interfere with esterase activity rate calculations, or measured fluorescence.

To further investigate the potential impact of protein product in the presence of an excipient on the assay, the model enzyme PCL, purified mAb 1 and purified mAb 3 were incubated with water (no spike), or spiked with an excipient (PS20 or PS80). As indicated by FIG. 7 (bottom), the addition of PS20 or PS80 did not change the esterase activity rates for the protein samples tested compared to the controls with water added in place of polysorbate (no spike).

### 2.7. Assay Performance Characterization

To characterize the performance of the esterase activity assay, the specificity, precision (repeatability and intermediate precision), LOD and LOQ were evaluated. The three types of mAb samples used for evaluating assay specificity and precision were selected to cover a range of esterase activity rates (Table 2). In terms of assay specificity, the minimum difference in MU-C8 hydrolysis rates between mAb samples and buffer blanks (negative controls) exceeded 1 µM MU/h for all three mAbs tested. The relative standard deviation (RSD) values for repeatability and intermediate precision were <6% for all the three sample types tested.

TABLE 2

Assessment of assay specificity and precision using three types of mAb samples

| Sample | | | Esterase Activity Rate | | |
|---|---|---|---|---|---|
| Identity | Type^{∗} | ConceQntration (mg/mL) | Mean (µM MU/h)^{∗∗} | Minimum Difference (µM MU/h)^{∗∗∗} | RSD ^{∗∗∗∗} |
| mAb 1 | UFDF | 220 | 7.8 | 7.1 | 2.5 |
| mAb 6 | UFDF | 57 | 4.5 | 4.0 | 2.5 |
| mAb 2 | Affinity | 12 | 1.6 | 1.2 | 5.1 |

^{∗}Purification in-process pool sample type.
^{∗∗}Mean esterase activity rate for six plates over two assay sessions with 10 replicate samples per plate.
^{∗∗∗}Minimum difference in hydrolysis rate as calculated using equation 2.
^{∗∗∗∗}RSD (%) for intermediate precision over separate assay sessions by two analysts (n=10 per plate; 3 plates per analyst and assay session; separate reagent preparation per analyst).

To further characterize the impact of different sample matrices on the assay performance, three buffers were selected to cover the potential range of matrix interferences: 50 mM tris acetate represented the matrix with minimum interference, whereas 50 mM histidine chloride and 200 mM arginine chloride represented histidine and arginine buffers with observable matrix interference (FIG. 6). The LOD and LOQ values were established for the esterase activity assay using these three buffers (Table 3).

TABLE 3

Determination of LOD and LOQ for esterase activity assay

| Buffer | | Esterase Activity Assay* | |
|---|---|---|---|
| Composition | Concentration (mM) | LOD (µM MU/h)^{∗∗} | LOQ (µM MU/h)^{∗∗∗} |
| Tris acetate | 50 | 0.2 | 0.5 |
| Histidine chloride | 50 | 0.3 | 0.8 |
| Arginine chloride | 200 | 0.3 | 0.8 |

^{∗}Assay LOD and LOQ were assessed using three plates and three types of buffers (n=12 per plate).
^{∗∗}LOD was calculated using twice the one-sided 95% Cl_{esterase activity rate} (equation 3).
^{∗∗∗}LOQ was calculated using six times the one-sided 95% Clₑₛₜₑᵣₐₛₑ _{activity rate} (equation 3).

### 2.8. UFDF Pool Sample Comparison Across Purification Processes

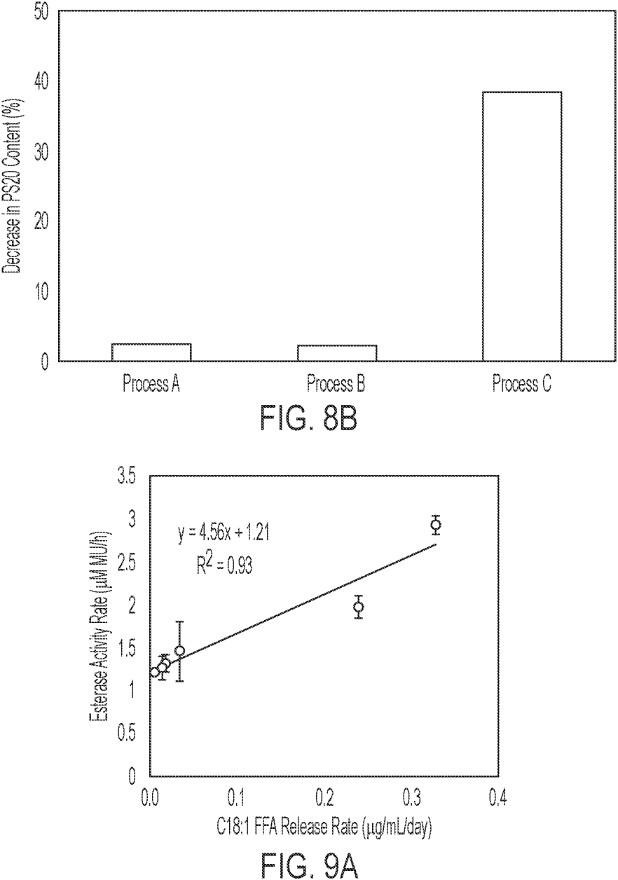

To assess the use of the assay for bioprocess development, esterase activity rates were compared for mAb5 samples purified to UFDF pools by three different purification processes: Process A, Process B and Process C (FIG. 8). The three processes varied in chromatography resins, loading density and pooling criteria. As indicated by measured esterase activity rates, Process C is hypothesized to be less efficient at the removal of esterases than Processes A or B due to the lower esterase activity rates measured in Processes A and B as compared to Process C. This hypothesis is further supported by the change in PS20 content at 40° C. measured by the HPLC-ELSD method for each of the three process. The same trend was observed as the esterase activity measurements: Process A and B showed minimal PS20 degradation, while Process C showed substantially higher PS20 degradation than Processes A or B (FIG. 8, right). In addition, the similarity in esterase activity rates for Process A and Process B corroborate with their similarity in PS20 content loss. These observations exemplify the relevance of this esterase activity assay in assessing polysorbate degradation risks from the purification process: it provides a rapid output (<3 hours for total assay turnaround time) to compare the effectiveness of different downstream processing conditions for removing residual esterases that degrade polysorbate.

### 2.9. FFA and Polysorbate Degradation Correlation

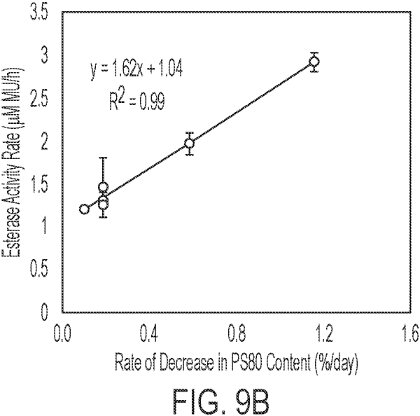

To investigate the use of the assay for predicting polysorbate degradation risks, rates of PS80 degradation and esterase activity in different pool samples for mAb 2 were compared (FIG. 9). The samples tested included purification in-process pools from multiple stages (affinity chromatography, ion exchange chromatography and UFDF) for two different purification processes applied to mAb 2. All samples were processed to the same formulation at 25 mg/ml for ease of comparison. PS80 degradation was measured by the increase in the major degradation product (C18:1 FFA) and by the decrease in the PS80 content. Positive correlations between the rates of C18:1 FFA release and esterase activity (FIG. 9, left) and between the rates of PS80 content decrease and esterase activity (FIG. 9, right) were observed.

### 3. Discussion

The results disclosed herein demonstrate the suitability of this esterase activity assay for assessing the risk of polysorbate degradation during bioprocess development in a rapid, high-throughput manner. The MU-C8 substrate was tested against a range of purification in-process pool and purified protein (UFDF pool) samples (FIGS. 2-5, 7-9). The purified samples (mAbs 1-5) had undergone chromatography steps to remove residual HCPs, and represent UFDF pools that would be processed into drug substance and drug product. Therefore, the samples tested here are expected to have lower levels of HCPs than the spiked samples tested by Jahn et al. The samples tested here also included multiple mAb products (four IgG1s and two IgG4s produced by CHO hosts, and an IgG4 produced by an E. coli host). During the early development of the esterase activity assay, the excitation wavelength and the concentrations of the assay running buffer components (tris chloride, Triton X-100 and gum arabic) were optimized (data not shown). The assay conditions tested here represent the optimized conditions and are different from those previously reported.

Based on the data collected using the esterase activity assay, it is apparent that the assay can detect the residual esterase activity in purification in-process (FIG. 9 and Table 2) and purified protein samples (FIGS. 2-5, 7-9). FIG. 3 demonstrates that there is reliable measurable activity in the UFDF pool samples that is distinguishable from background fluorescence (attributed to non-enzymatic hydrolysis), indicating the presence of enzyme(s) that are capable of hydrolyzing the carboxylic ester bond in MU-C8. The fluorescence traces for reaction raw rate and non-enzymatic hydrolysis show a high degree of reproducibility (FIG. 3), as indicated by the small, non-overlapping error bars representing standard deviation across duplicate samples. The additional fluorescence traces from FIG. 5 further demonstrate that the microplate-based esterase activity assay generated reliable and reproducible data within plates and across plates.

To support more rapid bioprocess development and efficient investigations, we aimed to shorten the assay incubation time reported by Jahn et al. (~24-300 hours) for the lipase assay, which uses Eppendorf tubes for the hydrolysis reactions. As shown in FIG. 3, the esterase activity assay offers a shorter incubation time (2 hours) and supports same day readout. The microplate-based format enables high-throughput screening of more samples and assay conditions (30 samples per plate run in duplicate consisting of up to seven different buffer blank matrices). This microplate format is also amenable to further efficiency gains through automation. To select the appropriate fluorogenic MU ester substrate, five MU esters were tested: MU-C8, MU-C10, MU-C12, MU-C16 and MU-C18:1. A variety of MU esters were selected to assess the effect of steric hindrance and carbon chain length on esterase activity. However, MU esters with shorter chain lengths were not selected because MU-C4 was shown to be sub-optimal substrates for lipases and phospholipases, and chromogenic substrates with C2 and C4 were previously shown to have poor responses across 33 hydrolytic enzymes (primarily lipases) tested.

Substrate specificity experiments shown in FIG. 2 indicated that MU-C8 is a broad and sensitive substrate to use for both model enzymes and purified protein samples. MU-C16 was found to be the least soluble substrate, which limited its concentration to 0.5 mM in these experiments. The MU-C18:1 substrate was included to understand implications of an oleic acid (C18:1) side chain present in PS80. Unlike MU-C8, this MU-C18:1 substrate did not show activity to any of the purified protein (mAbs 1-3) samples tested. Previous studies reported in literature have indicated that umbelliferone is a more stable fluorophore compared to 4-MU. However, those studies did not test biopharmaceutical samples. By contrast, our studies tested purified mAb samples generated using biopharmaceutically relevant processes, and our results indicate that MU-C8 can be utilized for an esterase activity assay with a high degree of reproducibility and across various sample types (Table 2).

In addition to testing a variety of MU ester substrates, we also tested a range of model enzymes and sample types. The three model enzymes were selected as positive controls for their connections to polysorbate degradation: (1) PCL represents a model lipase that degrades PS20 and PS80; (2) LPLA2 degrades PS20 and PS80 and was found in mAb formulations (9); (3) PLBL2 was identified as a residual H CP associated with PS20 degradation in a sulfatase drug product. The assay detected esterase activities in the model enzymes (PCL, LPLA2, and PLBL2) and purified proteins (mAbs 1-3) as shown in the substrate specificity (FIG. 2) and pH dependence (FIG. 5) experiments. Additionally, the inclusion of the E.coli-derived purified protein sample (mAb 3) demonstrates the capability of this assay to detect enzyme activity from a bacterial production system (FIG. 2).

The limited esterase activity observed for PLBL2 in this work concurs with recent findings indicating that PLBL2, also known as PLBD2, is unlikely to contribute towards PS degradation. As shown in FIG. 2, PLBL2 was significantly less active towards hydrolyzing the five MU esters tested than the other two enzymes (PCL and LPLA2)-20,000-fold higher PLBL2 concentration (400 µg/ml) was required to elicit MU ester hydrolysis relative to PCL and LPLA2 (both tested at 20 ng/ml).

In the presence of lipase inhibitor orlistat, the purified protein samples and model enzymes showed decreased esterase activity towards MU-C8 hydrolysis (FIG. 4). Orlistat was developed to inhibit pancreatic lipases and it exhibits different potencies towards different enzymes. In particular, orlistat significantly inhibited carboxylesterase 2 but not carboxylesterase 1. This lack of orlistat potency towards carboxylesterase 1 (CES1) is noteworthy because CES1 bears substantial sequence homology to two CHO carboxylesterases (CES-B1L and CES-1L) recently identified as the root cause for polysorbate degradation in a mAb formulation.

A separate orlistat incubation study was conducted to directly assess effect of orlistat on PS20 degradation in protein samples (mAbs 2, 6, and 7) by measuring the release of lauric acid, the primary FFA degradant from PS20 hydrolysis. Orlistat did not completely inhibit the hydrolysis of PS20 in these protein samples even when the orlistat concentration was increased from 0.2 µM to 20 µM (Table 1). Similarly, orlistat did not completely inhibit the hydrolysis of MU-C8 in the mAb 1 samples even when the orlistat concentration was increased from 0.5 µM to 10 µM (FIG. 4). In other orlistat incubation studies with a variety of purified mAb samples, we also observed a range in the efficacy of orlistat inhibition (data not shown). Taken together, these findings indicate that orlistat incubation mitigated PS20 degradation to varying extents in different protein samples. We hypothesize that (1) orlistat is not fully effective at inhibiting some of the CHO-derived enzymatic HCPs that degrade polysorbate; and (2) the identities and quantities of residual hydrolytic HCPs that are resistant or less sensitive to orlistat inhibition can differ across purified protein samples. Therefore, it is foreseeable that orlistat can inhibit some, but not all of the residual HCPs present in mAb 1 samples that can hydrolyze MU-C8. Likewise, it is foreseeable that orlistat can inhibit some, but not all of the residual HCPs present in mAb 2, mAb 6, and mAb 7 samples that can hydrolyze PS20.

The pH dependence experiments demonstrated a need for compromise between maximum fluorescence signal and reliable rate measurements. pH dependence curves shown in FIG. 5 indicate a sharp increase in activity rates from purified protein samples (mAb 2) between pH 7.5 and pH 8.5. The higher activity rates observed in mAb samples may be associated with higher enzyme activity in the alkaline pH range towards the MU-C8 substrate. Enzymes that were previously shown to degrade polysorbate in CHO-derived mAb samples (15, 20) may be more active in the alkaline pH range: LPL has elevated activity at pH 8 and higher (39); carboxylesterases have optimum activity at pH ~6.5-8.0 (14). The higher activity rates observed at increased pH can be attributed to increased base-mediated non-enzymatic hydrolysis of the substrate. The observed sharp increase in non-enzymatic hydrolysis rates may reduce assay sensitivity when operating at higher pH. However, even at pH 8.0, our studies indicate a clear difference in fluorescence between the sample (test) and enzyme blank (negative control) reaction wells. Therefore, the esterase activity rate for a given sample can be calculated by ensuring background fluorescence (i.e., signal from non-enzymatic hydrolysis in negative control) is consistently subtracted from the total fluorescence (test sample). The selected pH 8.0 for the esterase activity assay provides the optimum balance between sensitivity of low activity samples and increased non-enzymatic hydrolysis at more alkaline pH conditions.

At pH 8.0, the pH used for the esterase activity assay is higher than the typical pH in drug products. By contrast, the assays that detect FFA levels are set up to measure hydrolytic polysorbate degradation at the formulation pH; however, the degradation rates are so low that extended incubation times (> 1 week) are necessary to generate sufficiently high levels of FFAs to support reliable quantitation. In light of the difficulties in detecting trace levels of residual hydrolytic HCPs in purified samples, we prioritized selection of an assay pH that enhanced the esterase activity over the use of a representative formulation pH. Although we have observed higher enzyme activity at the higher assay pH of 8, a different trend is theoretically possible. For a given purified protein sample, the optimum pH for esterase activity is expected to depend on its enzyme profile. If the sample contained residual HCPs that have elevated esterase activities at lower pH, that sample would be expected to show a correspondingly lower pH optimum in the esterase activity assay. In such instances, the pH for the esterase activity assay may be lowered to improve assay sensitivity. However, such pH adjustments to the assay may not be necessary because the assay is primarily used to support bioprocess development (e.g., to rank purification schemes and conditions for polysorbate degradation risks) instead of formulation development. As shown by the bioprocess development example for mAb 5 (FIG. 8), the purification process that was least effective at removing hydrolytic HCPs (i.e., Process C) yielded the highest esterase activity rates (tested at pH 8.0) and the highest polysorbate degradation (tested at formulation pH <6.0).

The comprehensive assessment on buffer and salt species shown in FIG. 6 indicated that the majority of sample backgrounds are suitable for use in the esterase activity assay. The tested sample matrices did not negatively impact measured fluorescence and non-enzymatic hydrolysis rates. High levels of acetate and sulfate, commonly used in bioprocessing, can impact the measured fluorescence and may result in lower assay sensitivity. High levels of acetate, low levels of histidine and high levels of arginine increased base-mediated non-enzymatic hydrolysis of the substrate. The associated background fluorescence is addressed by ensuring that the non-enzymatic hydrolysis rate (k_{non-enzymatic hydrolysis}) is always subtracted from the reaction raw rate (k_{raw}) used for calculating the esterase activity rate. However, a higher non-enzymatic hydrolysis rate may lead to lower assay sensitivity when compared to samples with buffers that exhibit lower background fluorescence. In such instances, an examination of the raw data-to verify a clear difference between k_{raw} and k_{non-enzymatic} _{hydrolysis} will help determine if an additional sample processing step is necessary. For example, if clear separation between k_{raw} and k_{non-enzymatic hydrolysis} is not observed, sample adjustment measures (e.g., buffer exchange) can be implemented. The ranges tested for histidine were relevant to drug product formulations and may affect the ability to assay these samples without sample adjustment measures. For such formulations, if there is a need to increase assay sensitivity, the samples may be buffer exchanged to minimize background interference. However, our studies indicate that the majority of samples, even those containing histidine or arginine, can be assayed directly without a prior sample-conditioning step.

After optimizing the assay substrate (FIG. 2) and conditions (FIGS. 5 and 6), the assay was further tested to verify lack of protein or excipient interference (FIG. 7). High concentrations of mAb 4 (180 mg/ml) did not interfere with calculated esterase activity rates for model enzyme LPLA2 as demonstrated by the parallel activity lines between LPLA2 spiked with water and LPLA2 spiked with mAb 4 (FIG. 7, top left). The vertical difference between these two lines is ascribed to the esterase activity rate of mAb 4 itself, which showed a measurable activity rate on its own. This vertical difference remained constant throughout the concentration range tested and therefore, it did not affect the activity rate calculations for the model enzyme LPLA2. Additionally, fluorescence was not impacted by high concentrations of protein (FIG. 7, right) and esterase activity rates were not impacted by excipients, such as sucrose (data not shown), PS20 and PS80 (FIG. 7, bottom), at ranges relevant to biopharmaceutical formulations.

The performance of the esterase activity assay was further assessed for specificity, precision, LOD and LOQ (Tables 2 and 3). The characterization studies demonstrate assay specificity and precision (<6% RSD). As expected, the LOD and LOQ values for the histidine and arginine buffers associated with higher matrix interference (FIG. 6) were higher than for tris acetate (50 mM). Nonetheless, LOD and LOQ values for the esterase activity assay in all three buffers tested were relatively low (<1 µM MU/h; Table 3) compared with the typical assay output range (>1 µM MU/h; FIGS. 2, 4-9).

Finally, the assay was demonstrated to be suitable for assessing the risk of polysorbate degradation in representative bioprocess conditions. Specifically, the assay was compared against two conventional (and more time-consuming) methods for quantifying polysorbate degradation during bioprocess development-one that measures polysorbate content (6) and one that measures the FFA polysorbate degradants (8). The esterase activity assay was successful in identifying the bioprocesses that resulted in lower relative risk of PS20 degradation (FIG. 8). The esterase activity rates also positively correlated with PS80 degradation rates measured by both of the conventional methods (FIG. 9). Additionally, we have found a similar positive correlation for other mAbs where polysorbate degradation has been observed (data not shown). Taken together, these investigations show that this esterase activity assay can provide rapid assessment of polysorbate degradation risk during bioprocess development and non-routine investigations (e.g., troubleshooting during technology transfers). In spite of the positive correlation observed between the rates of esterase activity and polysorbate degradation, there are theoretical limitations to this esterase activity assay. The primary strength of this assay- the use of the fluorogenic MU-C8 substrate to provide an easier and faster assay readout-also represents its main weakness. MU-C8 is not PS20 or PS80, the surfactants typically used in drug products. Residual HCPs may differ in their hydrolytic activities towards the carboxylic ester bonds in MU-C8 versus PS20 or PS80. Likewise, the pH 8.0 used in this assay may enhance detection of esterase activities in purification samples, but it is not representative of the lower pH used in parenteral drug products.

To compensate for the theoretical limitations of the esterase activity assay, a two-stage approach may be applied during bioprocess development: (1) screen purification schemes/conditions to identify the most promising purification processes using the esterase activity assay; and (2) directly evaluate polysorbate degradation in the purified product for the top bioprocess options to select the final downstream process using an FFA-based assay. In the first stage, by measuring the residual levels of HCPs that degrade MU-C8, the esterase activity assay supports a key goal in bioprocess development of optimizing downstream processing for HCP removal. The esterase activity assay can screen a large number of samples to select a subset for subsequent testing by the FFA-based assay (in final formulation conditions with polysorbate as substrate). In the second stage, by directly measuring and correlating the extent of polysorbate degradation to the esterase activity rates obtained during the first stage, the relevance and correlation of the esterase activity assay for assessing polysorbate degradation risks can be determined (as shown by the examples in FIGS. 8 and 9). In this manner, the two-stage approach addresses the limitations of each tool for assessing polysorbate degradation while leveraging the strengths of each assay for guiding bioprocess development.

### Abbreviations

α: Calculated conversion factor of fluorescence signal to concentration of MU in RFU/µM
MU
C18:1: Oleic acid
CES1: Carboxylesterase 1
CES-1L: Carboxylesterase 1-like
CES-B1L: Carboxylesterase B-1-like
CHO: Chinese hamster ovary
Clesterase activity rate: Confidence interval for esterase activity rate
DMSO: Dimethyl sulfoxide
E. coli: Escherichia coli
ELSD: Evaporative light scattering detector
FFA: Free fatty acid
HCP: Host cell protein
HPLC: High performance liquid chromatography
k_{non-enzymatic hydrolysis}: Reaction rate of enzyme blank in RFU/h
k_{raw}: Reaction raw rate of sample in RFU/h
LOD: Limit of detection
LOQ: Limit of quantitation
LPL: Lipoprotein lipase
LPLA2: Lysosomal phospholipase A2
mAb: monoclonal antibody
MU: 4-methylumbelliferone
MU-C7: 4-methylumbelliferyl heptanoate MU-C8: 4-methylumbelliferyl caprylate MU-C10: 4-methylumbelliferyl decanoate
MU-C12: 4-methylumbelliferyl dodecanoate
MU-C16: 4-methylumbelliferyl palmitate
MU-C18:1: 4-methylumbelliferyl oleate
n: Sample size
PCL: Pseudomonas cepacia lipase
PDA: Photodiode array
PLBL2/PLDB2: Phospholipase B-like 2
PPL: Porcine pancreatic lipase
PS20: Polysorbate 20
PS80: Polysorbate 80
RFU: Relative fluorescence units
RSD: Relative standard deviation
SD: Standard deviation
UFDF: Ultrafiltration diafiltration
UHPLC: Ultra high performance liquid chromatography

1. An assay for determining enzymatic activity of host cell proteins (HCPs) in a sample, wherein the HCPs comprises hydrolase, the assay comprising the steps of:
a) obtaining a reaction mixture in a microplate, wherein the reaction mixture comprises: the sample, a reaction buffer, and a 4-methylumbelliferone carboxylate ester as a fluorogenic substrate;
b) obtaining a negative control;
c) exposing the reaction mixture and the negative control to fluorescence signal;
d) monitoring conversion of the fluorogenic substrate from a non-fluorescent state to a fluorescent product in the reaction mixture resulting from exposure to the fluorescence signal, wherein the fluorescent product is 4-methylumbelliferone (MU); and
e) determining and quantifying the HCP enzymatic activity based on conversion of the fluorogenic substrate in step d).

2. The assay of claim 1, wherein the sample comprises two or more different HCPs.

3. The assay of claims 1 or 2, wherein the HCP enzymatic activity in step e) represents the collective activity of two or more HCPs in the sample.

4. The assay of any one of claims 1-3, wherein the reaction mixture comprises at least two different fluorogenic substrates.

5. The assay of any one of claims 1-4, wherein the HCPs include esterases.

6. The assay of any one of claims 1-5 wherein the HCPs include carboxylic ester hydrolases, and wherein the HCPs optionally include lipases and carboxylesterases.

7. The assay of any one of claims 1-6, wherein the fluorogenic substrate has a carbon chain length of 8, 10, 12, 16 and/or 18.

8. The assay of any one of claims 1-6, wherein the fluorogenic substrate is 4-methylumbelliferyl caprylate (MU-C8).

9. The assay of any one of claims 1-6, wherein the fluorogenic substrate is 4-methylumbelliferyl decanoate (MU-C10).

10. The assay of any one of claims 1-9, wherein the sample comprises a product from a prokaryotic or eukaryotic host.

11. The assay of any one of claims 1-10, wherein the sample comprises a recombinant protein produced by a prokaryotic or eukaryotic host.

12. The assay of any one of claims 1-11, wherein the sample comprises a recombinant protein produced by bacterial or mammalian host.

13. The assay of any one of claims 1-12, wherein the sample comprises a recombinant protein that is based on an IgG format and is produced by a bacterial or mammalian host.

14. The assay of any one of claims 1-13, wherein the sample comprises a recombinant protein that is based on an IgG format and is produced by an E.coli or a Chinese Hamster Ovary (CHO) host.

15. The assay of any one of claims 1-14, wherein the sample comprises a recombinant protein selected from the group consisting of an IgG1 mAb, an IgG4 mAb, a bi-specific antibody; a mAb produced by a bacterial host, and a mAb produced by a mammalian host.

16. The assay of any one of claims 1-15, wherein the negative control is an enzyme blank.

17. The assay of any one of claims 1-16, wherein the fluorogenic substrate in the reaction mixture has a concentration of about 0.1-5 mM, about 0.1-4 mM, about 0.1-3 m M, about 0.1-2 mM, or about 0.5-1.0 mM.

18. The assay of any one of claims 1-17, wherein the sample is a chromatography purified pool sample.

19. The assay of any one of claims 1-18, wherein in step b), the sample is exposed to an increase of fluorescence signal using excitation and emission wavelengths of 300-400 nm and 400-500 nm, respectively, optionally about 355 nm and 460 nm, respectively.

20. The assay of any one of claims 1-19, wherein in step c), the sample is being incubated, optionally for about 1-5 hours, about 1-4 hours, about 1-3 hours, or about 2 hours.

21. The assay of any one of claims 1-20, wherein in step c), the sample is monitored every 5-15 minutes, or wherein the sample is optionally monitored every 10 minutes.

22. The assay of any one of claims 1-21, wherein the reaction mixture has a pH of about 4-9, about 5¬9, about 6-9, about 7-9, or about 8.

23. The assay of any one of claims 1-22, wherein the enzymatic activity is used to assess the level of hydrolytic activity towards polysorbate degradation in the sample.

24. The assay of any one of claims 1-23, wherein an output of the assay is used to compare and select purification processes to improve removal of hydrolytic HCPs.

25. An assay for determining enzymatic activity of host cell proteins (HCPs) in a sample, wherein the HCPs comprises hydrolase, and the assay comprises the steps:
a) obtaining a reaction mixture comprising the sample, a reaction buffer, and a fluorogenic substrate, wherein the fluorogenic substrate is 4-methylumbelliferone carboxylate ester, wherein the carboxylate ester of the 4-methylumbelliferone carboxylate ester comprises no more than ten carbons;
b) measuring the fluorescent signal at one or more time points; and
c) determining and quantifying the HCP enzymatic activity based on the measured fluorescence.

26. The assay of claim 25, wherein the carboxylate ester of the 4-methylumbelliferone carboxylate ester comprises no more than 8 carbons.

27. The assay of claim 25, wherein the 4-methylumbelliferone carboxylate ester is MU-C8.

28. The assay of claim 25, wherein the 4-methylumbelliferone carboxylate ester is MU-C10.

29. The assay of claim 25, wherein the HCP enzymatic activity determined and quantified in step c) represents the collective activity of two or more HCPs in the sample.

30. The assay of any one of claims 25-29, wherein the assay further comprises
a. obtaining a negative control comprising the same reaction buffer and fluorogenic substrate as the reaction mixture;
b. measuring the fluorescent signal of the negative control at the same one or more time points; and
c. determining and quantifying the HCP enzymatic activity by subtracting the amount of fluorescent signal observed in the negative control from the amount of fluorescent signal observed in the reaction mixture.

31. The assay of any one of claims 25-30, wherein the reaction mixture comprises at least two different fluorogenic substrates.

32. The assay of any one of claims 25-31, wherein the HCPs include esterases.

33. The assay of any one of claims 25-32 wherein the HCPs include carboxylic ester hydrolases, optionally the HCPs include lipases and carboxylesterases.

34. The assay of any one of claims 25-33, wherein the sample comprises a product from a prokaryotic or eukaryotic host.

35. The assay of any one of claims 25-34, wherein the sample comprises a recombinant protein produced by a prokaryotic or eukaryotic host.

36. The assay of any one of claims 25-35, wherein the sample comprises a recombinant protein produced by bacterial or mammalian host.

37. The assay of any one of claims 25-36, wherein the sample comprises a recombinant protein that is based on an IgG format and is produced by a bacterial or mammalian host.

38. The assay of any one of claims 25-37, wherein the sample comprises a recombinant protein that is based on an IgG format and is produced by an E.coli or a Chinese Hamster Ovary (CHO) host.

39. The assay of any one of claims 25-38, wherein the sample comprises a recombinant protein being selected from the group consisting of an IgG1 mAb, an IgG4 mAb, a bi-specific antibody; a mAb produced by a bacterial host, and a mAb produced by a mammalian host.

40. The assay of any one of claims 25-39, wherein the negative control is an enzyme blank.

41. The assay of any one of claims 25-40, wherein the fluorogenic substrate in the reaction mixture has a concentration of about 0.1-5 mM, about 0.1-4 mM, about 0.1-3 m M, about 0.1-2 mM, or about 0.5-1.0 mM.

42. The assay of any one of claims 25-41, wherein the sample is a chromatography purified pool sample.

43. The assay of any one of claims 25-42, wherein in step b), the sample is exposed to an increase of fluorescence signal using excitation and emission wavelengths of 300-400 nm and 400-500 nm, respectively, optionally about 355 nm and about 460 nm, respectively.

44. The assay of any one of claims 25-43, wherein in step c), the sample is incubated, optionally for about 2 hours, about 1-5 hours, about 1-4 hours, or about 1-3 hours.

45. The assay of any one of claims 25-44, wherein in step c), the sample is monitored every 5-15 minutes, or wherein the sample is optionally monitored every 10 minutes.

46. The assay of any one of claims 25-45, wherein the reaction mixture has a pH of about 4-9, about 5¬9, about 6-9, about 7-9, or about 8.

47. The assay of any one of claims 25-46, wherein the enzymatic activity is used to assess the level of hydrolytic activity towards polysorbate degradation in the sample.

48. The assay of any one of claims 25-47, wherein an output of the assay is used to compare and select purification processes to improve removal of hydrolytic HCPs.

49. A composition comprising
(a) an aqueous assay sample comprising a protein preparation,
(b) an organic solvent comprising a reaction buffer, and at least one 4-methylumbelliferone carboxylate ester;
wherein the fluorogenic substrate is 4-methylumbelliferone carboxylate ester, and wherein the carboxylate ester of the 4-methylumbelliferone carboxylate ester comprises no more than ten carbon atoms.

50. A method of determining stability of a protein preparation comprising
a. obtaining a reaction mixture in a microplate, wherein the reaction mixture comprises: the sample, a reaction buffer, and a 4-methylumbelliferone carboxylate ester as a fluorogenic substrate;
b. obtaining a negative control;
c. exposing the reaction mixture and the negative control to a fluorescence signal;
d. monitoring conversion of the fluorogenic substrate from a non-fluorescent state to a fluorescent product in the reaction mixture resulting from exposure to the fluorescence signal, wherein the fluorescent product is 4-methylumbelliferone (MU); and
e. determining and quantifying the HCP enzymatic activity based on conversion of the fluorogenic substrate in step d).

51. A method of optimizing or selecting a protein purification process to improve removal of hydrolytic HCPs, said method comprising
a. obtaining a reaction mixture in a microplate, wherein the reaction mixture comprises: the sample, a reaction buffer, and a 4-methylumbelliferone carboxylate ester as a fluorogenic substrate;
b. obtaining a negative control;
c. exposing the reaction mixture and the negative control to fluorescence signal;
d. monitor conversion of the fluorogenic substrate from a non-fluorescent state to a fluorescent product in the reaction mixture resulting from exposure to the fluorescence signal, wherein the fluorescent product is 4-methylumbelliferone (MU); and
e. determining and quantifying the HCP enzymatic activity based on conversion of the fluorogenic substrate in step d).
